SEQUENCE LISTING (8 sequences)

SEQ ID NO: 1 - RNA, 20 nt, Artificial sequence
  synthetic
accacaggguagaaccacgg

SEQ ID NO: 2 - RNA, 21 nt, Artificial sequence
  synthetic
accacaggguagaaccacgga

SEQ ID NO: 3 - RNA, 22 nt, Artificial sequence
  synthetic
accacaggguagaaccacggac

SEQ ID NO: 4 - RNA, 23 nt, Artificial sequence
  synthetic
uaccacaggguagaaccacggac

SEQ ID NO: 5 - RNA, 21 nt, Artificial sequence
  synthetic
uaccacaggguagaaccacgg

SEQ ID NO: 6 - RNA, 7 nt, Artificial sequence
  synthetic
ccacagg

SEQ ID NO: 7 - RNA, 22 nt, Artificial sequence
  synthetic
uaccacaggguagaaccacgga

SEQ ID NO: 8 - RNA, 20 nt, Artificial sequence
  synthetic
uaccacaggguagaaccacg

(19) United States
(12) Patent Application Publication
(10) Pub. No.: US 2024/0200064 A1
(43) Pub. Date: Jun. 20, 2024

(54) GENE THERAPY FOR INFLAMMATORY CONDITIONS

(71) Applicants: Oslo Universitetssykehus HF, Oslo (NO); Universitetet i Oslo, Oslo (NO)

(72) Inventors: Jan E. Brinchmann, Oslo (NO); Tommy A. Karlsen, Moss (NO); Rua Nader Al-Modawi, Oslo (NO)

(21) Appl. No.: 18/286,673

(22) PCT Filed: Apr. 12, 2022
(86) PCT No.: PCT/IB2022/000210

§ 371 Date: Oct. 12, 2023

Related U.S. Application Data
(60) Provisional application No. 63/174,232, filed on Apr. 13, 2021.

(51) Int. Cl. C12N 15/113; A61P 19/02

(52) U.S. Cl. CPC C12N 15/113; A61P 19/02; C12N 2310/141; C12N 2310/315; C12N 2310/321; C12N 2310/322

(57) ABSTRACT

The present invention relates to the use of oligonucleotides to treat inflammatory diseases, and in particular to treat osteoarthritis and rheumatoid arthritis. The oligonucleotides preferably comprise the seed sequence 5′-CCACAGG-3′ (SEQ ID NO: 6) situated at positions 2 to 8 of the oligonucleotide.

### FIELD OF THE INVENTION

The present invention relates to the use of oligonucleotides to treat inflammatory diseases, and in particular to treat osteoarthritis and rheumatoid arthritis.

### BACKGROUND OF THE INVENTION

Inflammation, mediated in part through the key inflammatory cytokines interleukin 1 beta (IL1β) and tumor necrosis factor alpha (TNFα), is an important driver in the pathogenesis of osteoarthritis (OA), as it promotes the degradation of existing extracellular matrix (ECM) and inhibits synthesis of new ECM.

Recent research points to the involvement of microRNAs (miRNAs) in these processes, miRNAs are small non-coding RNAs that regulate gene expression. They are transcribed in the nucleus as long transcripts called primary miRNA which are cleaved by the ribonuclease III endonuclease Drosha to shorter hairpin transcripts known as precursor miRNA (pre-miRNA). The pre-miRNA is then exported to the cytoplasm by Exportin 5. In the cytoplasm the double-stranded short hairpin pre-miRNA is further processed by another endoribonuclease, Dicer, which cuts off the loop to generate a mature miRNA duplex that is 20-25 nucleotides (nt) long. The two strands of the duplex are named according to the side of the hairpin from which they derive: 5p and 3p. One of the strands is selected by an Argonaute (AGO) protein, and together they form the RNA-induced silencing complex (RISC). Within the RISC the miRNA binds to its target mRNA through base-pairing between the so-called seed sequence (nucleotide 2-8) of the miRNA and the 3′ UTR of the mRNA. This leads to degradation and/or translational repression of the mRNA.

miR-140 has been considered a cartilage specific miRNA since it was observed to be predominantly expressed in cartilaginous tissue during development. Knockout studies showed miR-140 to be protective against OA development. Both miR-140-5p and miR-140-3p are highly upregulated during in vitro chondrogenesis, and we previously showed that both strands are highly expressed in healthy cartilage, miR-140-3p higher than miR-140-5p.

We also showed that miR-140-5p was essential for SOX9 expression during in vitro chondrogenesis, and demonstrated anti-inflammatory protective effects of both miR-140-5p and miR-140-3p on chondrocytes in two different in vitro models simulating OA.

The repertoire of miRNAs is increasing in complexity as recent deep sequencing studies have revealed the existence of many sequence variations in addition to the canonical sequences. These variants are called isomiRs. The sequence variation can be an addition or a deletion of one or more nucleotides at the 5′ and/or 3′ ends giving rise to 5′ or 3′ isomiRs. A substitution of a nucleotide gives rise to polymorphic isomiRs. IsomiRs are generated by RNA editing, alternative Drosha or Dicer processing, exonuclease mediated nucleotide trimming and/or non-templated nucleotide addition. 5′ isomiRs will have a different seed sequence from the canonical miRNA, and this may alter target recognition considerably. Recently the miRNA and isomiR prevalence in briefly cultured articular chondrocytes was published by Haseeb and colleagues. miR-140-3p was found to have the highest number of isomiRs, and several of these were found at higher prevalence than the canonical miR-140-3p. Another recent report also showed that miR-140-3p isomiRs were functional and regulated many other genes than canonical miR-140-3p.

### SUMMARY OF THE INVENTION

The present invention relates to the use of nucleobase compounds to treat inflammatory diseases, and in particular to treat osteoarthritis and rheumatoid arthritis. Here we show how the canonical miR-140-3p and two of its most prevalent isomiRs, one 5′ and one 3′ isomiR, vary in their effect on mRNA expression in articular chondrocytes in an inflammation-induced model of OA. The results showed that the three miRNAs overlapped in their regulation of the same biological processes, all with a predominantly anti-inflammatory effect. The 5′ isomiR, which by far downregulated the greatest number of mRNAs, showed extensive downregulation of genes involved in a number of immune response pathways. Notably, 5′ isomiRs will have a different seed sequence compared to the canonical miR-140-3p. This difference may affect target recognition, and the compounds may thus be able to provide desired effects in relation to treatment of inflammatory diseases. To illustrate, the canonical miR-140-3p can be represented as UACCACAGGGUAGAACCACGG (SEQ ID NO:5) wherein the seed sequence is underlined. The 5′isomiRs like SEQ ID NO:3 can be represented as ACCACAGGGUAGAACCACGGAC (SEQ ID NO:3) wherein the seed sequence is underlined.

Accordingly, in some preferred embodiments, the present invention provides methods for treating or preventing an inflammatory disease comprising administering to a subject in need thereof one or more oligonucleotides, or a salt thereof, or a pharmaceutical agent that induces the production of the one or more oligonucleotides, wherein the one or more oligonucleotides is at least 80% identical to any one of SEQ ID NOs: 1, 2 or 3 with the proviso that the oligonucleotides comprise the seed sequence CCACAGG (SEQ ID NO:6) situated at positions 2-8 from the 5′ end of the oligonucleotide. In some preferred embodiments, the one or more oligonucleotides is at least 90% identical to any one of SEQ ID NOs: 1, 2 or 3. In some preferred embodiments, the inflammatory disease is selected from the group consisting of inflammatory HLA-related immune diseases, inflammatory arthritic diseases, and inflammatory auto-immune diseases. In some preferred embodiments, the one or more nucleotides consist of from 15 to 40 linked nucleobases. In some preferred embodiments, the one or more nucleotides is 100% identical to any of SEQ ID NOs:1, 2 or 3. In some preferred embodiments, the one or more oligonucleotides is a modified oligonucleotide. In some preferred embodiments, the oligonucleotide is an RNA. In some preferred embodiments, the oligonucleotide is an agomir. In some preferred embodiments, the agomir is double-stranded and comprises an antisense strand having 5′ and 3′ terminals, wherein the antisense strand has a cholesterol-modified 3′ terminal, a plurality of phosphorothioate internucleoside linkages at the 5′ and 3′ terminals, and a plurality of 2′ OMe modified bases. In some preferred embodiments, the one or more oligonucleotides is present in a pharmaceutical composition.

In some preferred embodiments, the one or more oligonucleotides is administered via intra-articular administration. In some preferred embodiments, the intra-articular administration delivers the one or more nucleotides to cartilage in the subject. In some preferred embodiments, administration of the one or more oligonucleotides results in down-regulation of the interferon gamma cascade in the tissue of a subject. In some preferred embodiments, administration of the one or more oligonucleotides results in down-regulation of the interferon gamma cascade in the tissue of a subject at the site of administration. In some preferred embodiments, administration of the one or more oligonucleotides results in down-regulation of HLA class II genes in the tissue of a subject. In some preferred embodiments, administration of the one or more oligonucleotides results in down-regulation of HLA class II genes in the tissue of a subject at the site of administration. In some preferred embodiments, administration of the one or more oligonucleotides results in down-regulation of HLA class I genes in the tissue of a subject. In some preferred embodiments, administration of the one or more oligonucleotides results in down-regulation of HLA class I genes in the tissue of a subject at the site of administration.

In some preferred embodiments, the subject has osteoarthritis. In some preferred embodiments, the administration of the one or more nucleotides ameliorates one or more symptoms of osteoarthritis in the subject.

In some preferred embodiments, the subject has rheumatoid arthritis. In some preferred embodiments, the administration of the one or more nucleotides ameliorates one or more symptoms of rheumatoid arthritis in the subject.

In other preferred embodiments, the present invention provides oligonucleotide compositions comprising one or more nucleotides, or salts thereof, or a pharmaceutical agent that induces the production of the one or more oligonucleotide, wherein the one or more oligonucleotides is at least 80% identical to any one of SEQ ID NOs:1, 2 or 3 with the proviso that the oligonucleotides comprise the seed sequence CCACAGG (SEQ ID NO:6) situated at positions 2-8 from the 5′ end of the oligonucleotide. In some preferred embodiments, the one or more oligonucleotides is at least 90% identical to SEQ ID NOs:1, 2 or 3. In some preferred embodiments, the oligonucleotide compositions are provided for use in treating an inflammatory disease. In some preferred embodiments, the inflammatory disease is selected from the group consisting of inflammatory HLA-related immune diseases, inflammatory arthritic diseases, and inflammatory auto-immune diseases. In some preferred embodiments, the one or more nucleotides consist of from 15 to 40 linked nucleobases. In some preferred embodiments, the one or more nucleotides consist of any one of SEQ ID NOs:1, 2 or 3. In some preferred embodiments, the one or more oligonucleotides is a modified oligonucleotide. In some preferred embodiments, the one or more oligonucleotides is present in a pharmaceutical composition. In some preferred embodiments, the oligonucleotide is an RNA. In some preferred embodiments, the oligonucleotide is an agomir. In some preferred embodiments, the agomir is double-stranded and comprises an antisense strand having 5′ and 3′ terminals, wherein the antisense strand has a cholesterol-modified 3′ terminal, a plurality of phosphorothioate internucleoside linkages at the 5′ and 3′ terminals, and a plurality of 2′ OMe modified bases.

In some preferred embodiments, the one or more oligonucleotides is administered via intra-articular administration or topical administration. In some preferred embodiments, the intra-articular administration delivers the one or more nucleotides to cartilage in the subject.

In some preferred embodiments, administration of the one or more oligonucleotides results in down-regulation of the interferon gamma cascade in the tissue of a subject. In some preferred embodiments, administration of the one or more oligonucleotides results in down-regulation of the interferon gamma cascade in the tissue of a subject at the site of administration. In some preferred embodiments, administration of the one or more oligonucleotides results in down-regulation of HLA class II genes in the tissue of a subject. In some preferred embodiments, administration of the one or more oligonucleotides results in down-regulation of HLA class II genes in the tissue of a subject at the site of administration. In some preferred embodiments, administration of the one or more oligonucleotides results in down-regulation of HLA class I genes in the tissue of a subject. In some preferred embodiments, administration of the one or more oligonucleotides results in down-regulation of HLA class I genes in the tissue of a subject at the site of administration.

In some preferred embodiments, the subject has osteoarthritis. In some preferred embodiments, the administration of the one or more nucleotides ameliorates one or more symptoms of osteoarthritis in the subject. In some preferred embodiments, the subject has rheumatoid arthritis. In some preferred embodiments, the administration of the one or more nucleotides ameliorates one or more symptoms of rheumatoid arthritis in the subject.

In some preferred embodiments, the present invention provides a sterile pharmaceutical composition suitable for intra-articular administration comprising an oligonucleotide represented by SEQ ID NOs: 1, 2 or 3, wherein the oligonucleotide is double-stranded and comprises at least 20 one phosphorothioate linkage and at least one 2′-Fluoro or 2′-Methoxy base.

In some preferred embodiments, the present invention provides a sterile pharmaceutical composition suitable for intra-articular administration comprising an oligonucleotide represented by SEQ ID NOs: 1, 2 or 3, wherein the oligonucleotide is double-stranded and comprises an antisense strand having 5′ and 3′ terminals, wherein the antisense strand has a cholesterol-modified 3′ terminal, a plurality of phosphorothioate internucleoside linkage at the 5′ and 3′ terminals, and a plurality of 2′ OMe modified bases.

In some preferred embodiments, the present invention provides a pharmaceutical composition suitable for topical administration comprising an oligonucleotide represented by SEQ ID NOs: 1, 2 or 3, wherein the oligonucleotide is double-stranded and comprises at least one phosphorothioate linkage and at least one 2′-Fluoro or 2′-Methoxy base.

In some preferred embodiments, the present invention provides a pharmaceutical composition suitable for topical administration comprising an oligonucleotide represented by SEQ ID NOs: 1, 2 or 3, wherein the oligonucleotide is double-stranded and comprises an antisense strand having 5′ and 3′ terminals, wherein the antisense strand has a cholesterol-modified 3′ terminal, a plurality of phosphorothioate internucleoside linkages at the 5′ and 3′ terminals, and a plurality of 2′ OMe modified bases.

In some preferred embodiments, the present invention provides a method for treatment of inflammatory skin disease such as psoriasis, dermatitis (eczema), rosacea, or seborrheic dermatitis, comprising the step of topical administration of a suitable pharmaceutical composition, e.g. ointments or creams etc., containing an effective dose of an oligonucleotide represented by SEQ ID NOs: 1, 2 or 3, wherein, optionally, the oligonucleotide comprises at least one phosphorothioate internucleoside linkage and at least one 2′-Fluoro or 2′-Methoxy base.

In other embodiments, we provide nucleic acid vectors able to express an oligonucleotide represented by SEQ ID NOs: 1, 2 or 3. Such vectors may produce the desired oligonucleotides when absorbed by or transduced into human cells.

We also provide artificial oligonucleotides 80% identical to any one of SEQ ID NOs: 1, 2 or 3 with the proviso that the oligonucleotides comprise the seed sequence CCACAGG (SEQ ID NO:6) situated at positions 2-8 from the 5′ end of the oligonucleotide, wherein the oligonucleotide comprises at least one phosphorothioate internucleoside linkage and/or at least one 2′-Fluoro or 2′-Methoxy base. Accordingly, we provide oligonucleotides comprising SEQ ID NO: 1 and the seed sequence CCACAGG (SEQ ID NO:6) situated at positions 2-8 from the 5′ end of the oligonucleotide, wherein one or more of the bases comprise a stabilizing modification and/or the oligonucleotide comprises one or more stabilizing internucleoside linkages, e.g. phosphorothioate internucleoside linkages.

The claimed oligonucleotides herein may comprise a terminal thiophosphate. For example, such oligonucleotides may comprise a 3′ terminal thiophosphate and/or a 5′ terminal thiophosphate.

### BRIEF DESCRIPTION OF THE DRAWINGS

FIGS. 1A-C presents data relating to upregulation of OA mediators in response to IL1B and TNFα. (A) The sequences of the three miRNAs. (B) RT-qPCR analysis of IL1B, IL6, IL8, and MMP13 mRNA levels in unstimulated and IL1β and TNFα-stimulated conditions in chondrocytes from 3 OA donors. Error bars represent a 95% confidence interval from technical triplicates. (C) Western blot analysis of IL6 and IL8 protein levels in unstimulated and IL1β and TNFα-stimulated conditions in the same donors. All samples derive from the same experiment and the blots were processed in parallel. β-actin (ACTB) was used as loading control. D1=donor 1, D2=donor 2, D3=donor 3. ND=not detected.

FIG. 2 presents data related to increased levels of miRNAs following transfection. RT-qPCR analysis of canonical miR-140-3p, 5′ isomiR, and 3′ isomiR levels in chondrocytes from 3 donors after transfection. Error bars represent a 95% confidence interval from technical triplicates. IsomiRs were quantified with canonical miR-140-3p Taqman assay. D1=donor 1, D2=donor 2, D3=donor 3

FIG. 3A-B presents data demonstrating that canonical miR-140-3p and 3′ isomiR counteracted IL1β and TNFα-induced inflammation, while 5′ isomiR showed opposing tendencies. (A) RT-qPCR analysis of IL1B, IL6, and IL8 mRNA levels after transfection of the three miRNAs. Error bars represent a 95% confidence interval from technical triplicates. (B) Western blot analysis of IL6 and IL8 in the same three donors. All samples derive from the same experiment and the blots were processed in parallel. ACTB was used as loading control. D1=donor 1, D2=donor 2, D3=donor 3. ND=not detected.

FIGS. 4A-B presents RNA-sequencing data that demonstrates many immunological processes are targeted by all three miRNAs (A) Go-term analysis of the downregulated genes by canonical miR-140-3p, 5′ isomiR, and 3′ isomiR. The numbers in the figure are the FDR values for each term and the size of the blue dots correlates with significance (B) Venn diagram illustrating the number of common regulated genes by the miRNAs; downregulated and upregulated genes are shown separately.

FIG. 5 present data related to RT-qPCR validation of RNA-seq data. RT-qPCR analysis of IFNB, IFNG, DDX58 MX1, IRF1,2 and 7, CIITA, and after transfection of the three miRNAs. Error bars represent a 95% confidence interval from technical triplicates. GAPDH was used as endogenous control. D1=donor 1, D2=donor 2, D3=donor 3. ND=not detected.

FIG. 6 shows a western blot analysis of interferon beta and gamma after transfection with canonical miR-140-3p, 5′-isomiR and 3′-isomiR. All three miRNAs led to some downregulation of the proteins compared to negative control. However, 5′isomiR exhibited the most potent and consistent downregulation of the proteins among all donors. Protein was isolated from the same donors/the same cell samples previously used in the experiments to run the gene analysis.

FIGS. 7A-G present data demonstrating the effect of 5′ isomiR agomiR on inflammation in mouse knees following rupture of the anterior cruciate ligament for the following makers: (A) Il1b; (B) Zbp1; (C) Irf1; (D) Ifnb1; (E) Ifng; (F) Ciita; (G) Adamts4. Y axis values are mean of three biological triplicates, each of them based on three technical triplicates, relative to the internal control GAPDH. Invivo=Invivofectamine.

### DEFINITIONS

Unless defined otherwise, all technical and scientific terms used herein have the same meaning as is commonly understood by one of skill in the arts to which the claimed subject matter.

As used herein, the singular forms “a,” “an” and “the” include plural referents unless the context clearly dictates otherwise.

As used herein, “subject” means a human or non-human animal selected for treatment or therapy.

As used herein, “in need thereof” means a subject identified as in need of a therapy or treatment.

As used herein, “administering” means providing a pharmaceutical agent or composition to a subject, and includes, but is not limited to, administering by a medical professional and self-administering. As used herein, “parenteral administration,” means administration through injection or infusion. Parenteral administration includes, but is not limited to, subcutaneous administration, intravenous administration, intraarticular or intramuscular administration.

As used herein, “subcutaneous administration” means administration just below the skin.

As used herein, “intravenous administration” means administration into a vein.

As used herein, “therapy” means a disease treatment method.

As used herein, “treatment” means the application of one or more specific procedures used for the cure or amelioration of a disease. In some embodiments, the specific procedure is the administration of one or more pharmaceutical agents.

As used herein, “amelioration” means a lessening of severity of at least one indicator of a condition or disease. In some embodiments, amelioration includes a delay or slowing in the progression of one or more indicators of a condition or disease. The severity of indicators may be determined by subjective or objective measures which are known to those skilled in the art.

As used herein, “prevention” refers to delaying or forestalling the onset or development or progression of a condition or disease for a period of time, including weeks, months, or years.

As used herein, “therapeutic agent” means a pharmaceutical agent used for the cure, amelioration or prevention of a disease.

As used herein, “dosage unit” means a form in which a pharmaceutical agent is provided. In some embodiments, a dosage unit is a vial containing lyophilized oligonucleotide. In some embodiments, a dosage unit is a vial containing reconstituted oligonucleotide.

As used herein, “therapeutically effective amount” refers to an amount of a pharmaceutical agent that provides a therapeutic benefit to an animal.

As used herein, “pharmaceutical composition” means a mixture of substances suitable for administering to a subject that includes a pharmaceutical agent. For example, a pharmaceutical composition may comprise a modified oligonucleotide and a sterile aqueous solution.

As used herein, “pharmaceutical agent” means a substance that provides a therapeutic effect when administered to a subject.

As used herein, “active pharmaceutical ingredient” means the substance in a pharmaceutical composition that provides a desired effect.

As used herein, “targeting” means the process of design and selection of nucleobase sequence that will hybridize to a target nucleic acid and induce a desired effect.

As used herein, “targeted to” means having a nucleobase sequence that will allow hybridization to a target nucleic acid to induce a desired effect. In some embodiments, a desired effect is reduction of a target nucleic acid.

As used herein, “modulation” means to a perturbation of function or activity. In some embodiments, modulation means an increase in gene expression. In some embodiments, modulation means a decrease in gene expression.

As used herein, “expression” means any functions and steps by which a gene's coded information is converted into structures present and operating in a cell.

As used herein, “nucleobase sequence” means the order of contiguous nucleobases, in a 5′ to 3′ orientation, independent of any sugar, linkage, and/or nucleobase modification.

As used herein, “contiguous nucleobases” means nucleobases immediately adjacent to each other in a nucleic acid.

As used herein, “percent identity” means the number of nucleobases in first nucleic acid that are identical to nucleobases at corresponding positions in a second nucleic acid, divided by the total number of nucleobases in the first nucleic acid. Percent identity between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, “substantially identical” used herein may mean that a first and second nucleobase sequence are at least 60%, at least 65%, at least 70%, at least 75%, at least 80%, at least 85%, at least 90%, at least 95%, at least 97%, at least 98% at least 99%, or 100%, identical over a region of 8, 9, 10, 11, 12, 13, 14, 15, 16, 17, 18, 19, 20, 21, 22, 23, 24, 25, 30, 35, or 40 or more nucleobases.

As used herein, “hybridize” means the annealing of complementary nucleic acids that occurs through nucleobase complementarity.

As used herein, “mismatch” means a nucleobase of a first nucleic acid that is not capable of pairing with a nucleobase at a corresponding position of a second nucleic acid.

As used herein, “identical” means having the same nucleobase sequence.

As used herein, “oligomeric compound” means a compound comprising a polymer of linked monomeric subunits.

As used herein, “oligonucleotide” means a polymer of linked nucleosides, each of which can be modified or unmodified, independent from one another.

As used herein, the term “seed sequence” refers to a seven base sequence situated at positions 2-8 from the 5′-end of an miRNA oligonucleotide.

As used herein, “naturally occurring internucleoside linkage” means a 3′ to 5′ phosphodiester linkage between nucleosides.

As used herein, “natural sugar” means a sugar found in DNA (2′-H) or RNA (2′-OH).

As used herein, “natural nucleobase” means a nucleobase that is unmodified relative to its naturally occurring “internucleoside linkage” means a covalent linkage between adjacent nucleosides.

As used herein, “linked nucleosides” means nucleosides joined by a covalent linkage. As used herein, “nucleobase” means a heterocyclic moiety capable of non-covalently pairing with another nucleobase.

As used herein, “nucleoside” means a nucleobase linked to a sugar.

As used herein, “nucleotide” means a nucleoside having a phosphate group covalently linked to the sugar portion of a nucleoside.

As used herein, “modified oligonucleotide” means an oligonucleotide having one or more modifications relative to a naturally occurring terminus, sugar, nucleobase, and/or internucleoside linkage.

As used herein, “modified internucleoside linkage” means any change from a naturally occurring internucleoside linkage.

As used herein, “phosphorothioate internucleoside linkage” means a linkage between nucleosides where one of the non-bridging atoms is a sulfur atom.

As used herein, “modified sugar” means substitution and/or any change from a natural sugar.

As used herein, “modified nucleobase” means any substitution and/or change from a natural nucleobase.

As used herein, “5-methylcytosine” means a cytosine modified with a methyl group attached to the 5′ position.

As used herein, “2′-O-methyl sugar” or “2′-O-Me sugar” means a sugar having an O-methyl modification at the 2′ position.

As used herein, “2′-O-methoxyethyl sugar” or “2′-MOE sugar” means a sugar having a O-methoxyethyl modification at the 2′ position.

As used herein, “2′-O-fluoro” or “2′-F” means a sugar having a fluoro modification of the 2′ position.

As used herein, “bicyclic sugar moiety” means a sugar modified by the bridging of two non-geminal ring atoms.

As used herein, “2′-O-methoxyethyl nucleoside” means a 2′-modified nucleoside having a 2′-O-methoxyethyl sugar modification.

As used herein, “2′-fluoro nucleoside” means a 2′-modified nucleoside having a 2′-fluoro sugar modification.

As used herein, “2′-O-methyl” nucleoside means a 2′-modified nucleoside having a 2′-O-methyl sugar modification.

As used herein, “bicyclic nucleoside” means a 2′-modified nucleoside having a bicyclic sugar moiety. As used herein, “motif” means a pattern of modified and/or unmodified nucleobases, sugars, and/or internucleoside linkages in an oligonucleotide.

As used herein, a “fully modified oligonucleotide” means each nucleobase, each sugar, and/or each internucleoside linkage is modified.

As used herein, a “uniformly modified oligonucleotide” means each nucleobase, each sugar, and/or each internucleoside linkage has the same modification throughout the modified oligonucleotide.

As used herein, a “stabilizing modification” means a modification to a nucleoside that provides enhanced stability to a modified oligonucleotide, in the presence of nucleases, relative to that provided by 2′-deoxynucleosides linked by phosphodiester internucleoside linkages. For example, in some embodiments, a stabilizing modification is a stabilizing nucleoside modification. In some embodiments, a stabilizing modification is a internucleoside linkage modification.

As used herein, a “stabilizing nucleoside” means a nucleoside modified to provide enhanced nuclease stability to an oligonucleotide, relative to that provided by a 2′-deoxynucleoside. In one embodiment, a stabilizing nucleoside is a 2′-modified nucleoside.

As used herein, a “stabilizing internucleoside linkage” means an internucleoside linkage that provides enhanced nuclease stability to an oligonucleotide relative to that provided by a phosphodiester internucleoside linkage. In one embodiment, a stabilizing internucleoside linkage is a phosphorothioate internucleoside linkage.

### DETAILED DESCRIPTION OF THE INVENTION

The present invention relates to the use of oligonucleotides to treat inflammatory diseases, and in particular to treat osteoarthritis and rheumatoid arthritis.

Osteoarthritis (OA) is the most common degenerative disease. OA causes a large economical burden on the society and limited mobility, pain and an overall reduced life quality and mental health for the patient. Inflammation is believed to be one of the main drivers behind the pathogenesis of OA. microRNAs (miRNAs) are small non-coding RNAs that are potent gene regulators.

miR-140 is considered a cartilage specific miRNA and has a central role in cartilage development. There are two canonical miR-140; miR-140-5p and miR-140-3p (UACCACAGGGUAGAACCACGG; SEQ ID NO:5). Recent evidence shows that miR-140-3p, and not -5p is the most prevalent form of miR-140 in primary chondrocytes, and the most abundant of all chondrocyte-expressed miRNAs. Moreover miR-140-3p has the highest number of isoforms, so-called isomiRs, many of which are expressed at higher levels than the canonical sequence. Here we investigated the role of miR-140-3p and two of its most prevalent isomiRs; a 5′ isomiR (ACCACAGGGUAGAACCACGGAC, SEQ ID NO:3) and a 3′ isomiR (UACCACAGGGUAGAACCACGGAC, SEQ ID NO:4) in an inflammation-induced model of OA and show how all three miRNAs downregulate many of the same genes involved in key immune responses and inflammatory cascades. The 5′ isomiR (SEQ ID NO:3) showed a much greater target spectrum compared to the other two miRNAs. See Experimental Section below. Specifically, as compared to miR-140-3p, the 5′ isomiR provides a comprehensive immunosuppressive effect, including down-regulation of the interferon gamma and beta cascades and the IL1 beta cascade and down-regulation of all HLA class II genes and moderate down-regulation also of class I, and does not have the cartilage-building effect that miR-140-3p has been shown to have.

Accordingly, the present disclosure provides oligonucleotides, such as modified oligonucleotides, wherein the oligonucleotides, or a salt thereof, comprise a nucleobase sequence at least 80% identical to one of the following sequences:

(SEQ ID NO: 1)
ACCACAGGGUAGAACCACGG
(SEQ ID NO: 2)
ACCACAGGGUAGAACCACGGA,
(SEQ ID NO: 3)
ACCACAGGGUAGAACCACGGAC.

In some preferred embodiments, the oligonucleotides, or a salt thereof, comprise a nucleobase sequence at least 90% identical to SEQ ID NO:1, 2 or 3. In some preferred embodiments, the oligonucleotides, or a salt thereof, comprise a nucleobase sequence at least 95% identical to SEQ ID NOs.: 1, 2 or 3. In some preferred embodiments, the oligonucleotides, or a salt thereof, comprise the following seed sequence at positions 2 to 8 (numbered from the 5′ end of the oligonucleotide) of the oligonucleotide: CCACAGG (SEQ ID NO:6). In some preferred embodiments, the nucleobase is position 1 is an A. In some embodiments, the oligonucleotides, or salts thereof, do not comprise a 5′ terminal uracil residue. In some preferred embodiments, the oligonucleotides have an adenosine nucleobase at position 21 (numbered from the 5′ end of the oligonucleotide). In some preferred embodiments, the oligonucleotides have an adenosine nucleobase at position 21 and a cytosine nucleobase at position 22 (numbered from the 5′ end of the oligonucleotide). In some preferred embodiments, the oligonucleotides consist of from 15 to 40 linked nucleobases. In still further preferred embodiments, the oligonucleotides consist of the linked nucleobase sequence corresponding to any one of SEQ ID NOs: 1, 2 and 3.

In some embodiments, an oligonucleotide consists of 15 to 30 linked nucleobases. In some embodiments, an oligonucleotide consists of 19 to 24 linked nucleobases. In some embodiments, an oligonucleotide consists of 21 to 24 linked nucleobases. In some embodiments, an oligonucleotide consists of 22 linked nucleobases. In some embodiments, the oligonucleotide consists of 15, 16, 17, 18, 19, 20, 21, 22, 23, 24, 25, 26, 27, 28, 29, 30, 31, 32, 33, 34, 35, 36, 37, 38, 39, or 40 linked nucleobases. In some embodiments, the oligonucleotide consists of 15, 16, 17, 18, 19, 20, 21, 22, 23, 24, 25, 26, 27, 28, 29, or 30 linked nucleobases. In some embodiments, the oligonucleotide consists of 15, 16, 17, 18, 19, 20, 21, 22, 23, 24, or 25 linked nucleobases. In some embodiments, the oligonucleotide comprises a nucleobase sequence comprising at least 16, at least 17, at least 18, at least 19, at least 20, at least 21, or at least 22, or at least 23 contiguous nucleobases of a nucleobase sequence of any one of SEQ ID NOs: 1 to 3.

In some embodiments, the nucleobase sequence of the oligonucleotide has no more than three mismatches compared to the nucleobase sequences of any one of SEQ ID NOs: 1, 2 or 3. In some embodiments, the nucleobase sequence of the oligonucleotide has no more than two mismatches compared to the nucleobase sequences of any one of SEQ ID NOs: 1, 2 or 3. In some embodiments, the nucleobase sequence of the oligonucleotide has no more than one mismatch compared to the nucleobase sequence of any one of SEQ ID NOs:1, 2, or 3.

In some embodiments, the nucleobase sequence of the oligonucleotide has no more than three mismatches compared to the nucleobase sequences of SEQ ID NOs:1-3, with the proviso that the oligonucleotides comprise the seed sequence SEQ ID NO:6 at positions 2 to 8 (numbered from the 5′ end of the oligonucleotide) of the oligonucleotide. In some embodiments, the nucleobase sequence of the oligonucleotide has no more than two mismatches compared to the nucleobase sequences of SEQ ID NOs:1-3, with the proviso that the oligonucleotides comprise the seed sequence SEQ ID NO:6 at positions 2 to 8 (numbered from the 5′ end of the oligonucleotide) of the oligonucleotide. In some embodiments, the nucleobase sequence of the oligonucleotide has no more than one mismatch compared to the nucleobase sequences of SEQ ID NO:1-3, with the proviso that the oligonucleotides comprise the seed sequence SEQ ID NO:6 at positions 2 to 8 (numbered from the 5′ end of the oligonucleotide) of the oligonucleotide.

In some embodiments, the nucleobase sequence of the oligonucleotide has no more than two mismatches compared to the nucleobase sequence SEQ ID NO:3, with the proviso that the oligonucleotides have an adenosine nucleobase at position 21 of SEQ ID NO:3 and a cytosine nucleobase at position 22 of SEQ ID NO:3. In some embodiments, the nucleobase sequence of the oligonucleotide has no more than one mismatch compared to the nucleobase sequence SEQ ID NO:3, with the proviso that the oligonucleotides have an adenosine nucleobase at position 21 of SEQ ID NO:3 and a cytosine nucleobase at position 22 of SEQ ID NO:3.

In some embodiments, the nucleobase sequence of the oligonucleotide has no mismatches compared to the nucleobase sequence corresponding to SEQ ID NOs:1, 2 or 3. In each of these embodiments, the oligonucleotide can be a modified oligonucleotide. In some preferred embodiments, the oligonucleotides are RNAs.

Oligonucleotides of the present invention are intended to provide an anti-inflammatory effect able to provide a prophylactic or therapeutic treatment of arthritis, osteoarthritis or other inflammation-induced conditions if locally administered via intra-articular injections, intrathecal injections or by other topical applications. In some preferred embodiments, the oligonucleotides are provided as double-stranded molecules (e.g. with a complementary strand) which may be conjugated to a moiety facilitating cellular uptake. In some preferred embodiments, at least 90%, 95% or 100% of the bases in said double-stranded molecules are in the form of 2′-Fluoro or 2′-Methoxy bases. In some preferred embodiments, the terminal nucleotides in the double-stranded molecules comprise a phosphorothioate internucleoside linkage. Accordingly, in one preferred embodiment, there is provided a double-stranded oligonucleotide wherein one strand is represented by the any one of SEQ ID NOs: 1, 2 or 3, or sequences at least 80%, 90% or 95% identical to any one of SEQ ID NOs:1, 2, 3 and which comprise the seed sequence SEQ ID NO:6) and wherein all of the bases are either 2′-Fluoro or 2′-Methoxy bases, and wherein the complementary strand is conjugated to triantennary N-acetylgalactosamine at the 3′ terminal, and wherein the other terminal nucleotides comprise at least one phosphorothioate internucleoside linkage. Triantennary N-acetylgalactosamine is a moiety represented by the formula:

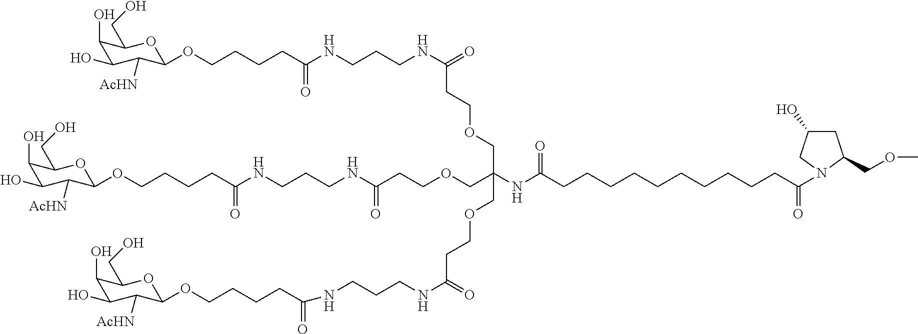 [SMILES: COC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some particularly preferred embodiments, the present disclosure provides a pharmaceutical composition suitable for intra-articular injection comprising an RNA oligonucleotide containing the seed sequence CCACAGG ((SEQ ID NO: 6), i.e. the sequence starting from nucleotide number 2 from the 5′ end to nucleotide 8). In some preferred embodiments, nucleotide 1 is an A. In some embodiments, the oligonucleotide is at least 80% identical to SEQ ID NOs: 1, 2 or 3. In some preferred embodiments, the oligonucleotides are 100% identical to any one of SEQ ID NOs: 1, 2 or 3.

In some preferred embodiments, the oligonucleotides are provided as double-stranded molecules (e.g. with a complementary strand) which may optionally be conjugated to a moiety facilitating cellular uptake. In some preferred embodiments, at least 90%, 95% or 100% of the bases in said double-stranded molecules are in the form of 2′-Fluoro or 2′-Methoxy bases. In some preferred embodiments, the terminal nucleotides in the double-stranded molecules comprise a phosphorothioate internucleoside linkage. In other preferred embodiment, the double-stranded RNA oligonucleotide is an agomir (GenePharma, Shanghai China). Agomirs are characterized in comprising an antisense strand with 3′ and 5′ terminals with one or more of the following following modifictions: 1) the 3′ terminal is modified with a cholesterol; 2) the 5′ terminal comprises phosphorothioate internucleoside linkages between the first and second nucleside and second and third nucleoside from the 5′ terminal; 3) the 3′ terminal comprises phosphorothioate internucleoside linkage between the first and second, second and third, third and fourth, and fourth and fifth nucleosides from the 3′ terminal; and 4) preferably all of the bases in the antisense strand are 2′-OMe modified bases.

Pharmaceutical compositions suitable for intra-articular injection may preferably be isotonic, sterile solutions with a pH in the range of 6 to 8. The RNA oligonucleotides may be formulated into micelles or liposomes for facilitating cellular uptake. However, they may also be electroporated into target cells in situ or uptake into target cells may be facilitated via other, novel means. Such pharmaceutical compositions may provide an anti-inflammatory effect able to provide a prophylactic or therapeutic treatment of arthritis or osteoarthritis.

In other preferred embodiments, the present disclosure provides a pharmaceutical composition suitable for topical administration comprising an RNA oligonucleotide containing the seed sequence CCACAGG ((SEQ ID NO:6), i.e., the sequence starting from nucleotide number 2 from the 5′ end to nucleotide 8). In some preferred embodiments, nucleotide 1 is an A. In some embodiments, the oligonucleotide is at least 80% identical to SEQ ID NOs: 1, 2 or 3. In some preferred embodiments, the oligonucleotides are 100% identical to any of SEQ ID NOs: 1, 2 or 3. Pharmaceutical compositions suitable for topical administration may be ointments, creams etc. allowing transdermal uptake of the RNA oligonucleotides. Such pharmaceutical compositions may provide an anti-inflammatory effect able to provide a prophylactic or therapeutic treatment of inflammatory skin conditions.

In other alternative and separate embodiments, the oligonucleotides may have the following sequences:

(SEQ ID NO: 4)
UACCACAGGGUAGAACCACGGAC,
(SEQ ID NO: 7)
UACCACAGGGUAGAACCACGGA,
and
(SEQ ID NO: 8)
UACCACAGGGUAGAACCACG

It is contemplated that these RNA oligonucleotides have therapeutic or prophylactic utility in relation to arthritis.

The present invention is not limited to the use of any particular oligonucleotide formats. Suitable nucleic acids include, but are not limited to, deoxyribonucleic acid (DNA), ribonucleic acid (RNA), modified DNA or RNA, peptide nucleic acid (PNA), morpholino, locked nucleic acid (LNA), glycol nucleic acid (GNA), threose nucleic acid (TNA), DNA containing phosphorothioate residues (S-oligos) and derivatives thereof, or any combination thereof.

In some embodiments, one or more additional nucleobases may be added to either or both of the 3′ terminus and 5′ terminus of an oligonucleotide in comparison to the nucleobases sequences set forth in any of SEQ ID NOs:1, 2 or 3. In some embodiments, the one or more additional linked nucleobases are at the 3′ terminus. In some embodiments, the one or more additional linked nucleosides are at the 5′ terminus. In some embodiments, two additional linked nucleosides are linked to a terminus. In some embodiments, one additional nucleoside is linked to a terminus. In each of these embodiments, the oligonucleotide can be a modified oligonucleotide.

In some embodiments, the oligonucleotide comprises one or more modified internucleoside linkages, modified sugars, or modified nucleobases, or any combination thereof. The nucleobase sequences set forth herein, including but not limited to those found in the Examples and in the sequence listing, are independent of any modification to the nucleic acid. As such, nucleic acids defined by a SEQ ID NO: may comprise, independently, one or more modifications to one or more sugar moieties, to one or more internucleoside linkages, and/or to one or more nucleobases. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases.

In some embodiments, at least one internucleoside linkage is a modified internucleoside linkage. In some embodiments, each internucleoside linkage is a modified internucleoside linkage. In some embodiments, a modified internucleoside linkage comprises a phosphorus atom. In some embodiments, a modified oligonucleotide comprises at least one phosphorothioate internucleoside linkage. In some embodiments, each internucleoside linkage of a modified oligonucleotide is a phosphorothioate internucleoside linkage. In some embodiments, a modified internucleoside linkage does not comprise a phosphorus atom. In some such embodiments, an internucleoside linkage is formed by a short chain alkyl internucleoside linkage. In some such embodiments, an internucleoside linkage is formed by a cycloalkyl internucleoside linkages. In some such embodiments, an internucleoside linkage is formed by a mixed heteroatom and alkyl internucleoside linkage. In some such embodiments, an internucleoside linkage is formed by a mixed heteroatom and cycloalkyl internucleoside linkages. In some such embodiments, an internucleoside linkage is formed by one or more short chain heteroatomic internucleoside linkages. In some such embodiments, an internucleoside linkage is formed by one or more heterocyclic internucleoside linkages. In some such embodiments, an internucleoside linkage has an amide backbone. In some such embodiments, an internucleoside linkage has mixed N, O, S and CH₂ component parts.

In some embodiments, at least one nucleobase of the modified oligonucleotide comprises a modified sugar. In some embodiments, each of a plurality of nucleosides comprises a modified sugar. In some embodiments, each nucleoside of the modified oligonucleotide comprises a modified sugar. In each of these embodiments, the modified sugar may be a 2′-O-methoxyethyl sugar, a 2′-fluoro sugar, a 2′-O-methyl sugar, or a bicyclic sugar moiety. In some embodiments, each of a plurality of nucleosides comprises a 2′-O-methoxyethyl sugar and each of a plurality of nucleosides comprises a 2′-fluoro sugar.

In some embodiments, the sugar-modified nucleosides can further comprise a natural or modified heterocyclic base moiety and/or a natural or modified internucleoside linkage and may include further modifications independent from the sugar modification. In some embodiments, a sugar modified nucleoside is a 2′-modified nucleoside, wherein the sugar ring is modified at the 2′ carbon from natural ribose or 2′-deoxyribose.

In some embodiments, a 2′-modified nucleoside has a bicyclic sugar moiety. In some such embodiments, the bicyclic sugar moiety is a D sugar in the alpha configuration. In some such embodiments, the bicyclic sugar moiety is a D sugar in the beta configuration. In some such embodiments, the bicyclic sugar moiety is an L sugar in the alpha configuration. In some such embodiments, the bicyclic sugar moiety is an L sugar in the beta configuration. In some embodiments, the bicyclic sugar moiety comprises a bridge group between the 2′ and the 4′-carbon atoms. In some such embodiments, the bridge group comprises from 1 to 8 linked biradical groups. In some embodiments, the bicyclic sugar moiety comprises from 1 to 4 linked biradical groups. In some embodiments, the bicyclic sugar moiety comprises 2 or 3 linked biradical groups. In some embodiments, the bicyclic sugar moiety comprises 2 linked biradical groups. Biradical groups are well known in the art.

In some embodiments, the modified oligonucleotide comprises at least one modified nucleobase. In some embodiments, the modified nucleobase is selected from 5-hydroxymethyl cytosine, 7-deazaguanine and 7-deazaadenine. In some embodiments, the modified nucleobase is selected from 7-deaza-adenine, 7-deazaguanosine, 2-aminopyridine and 2-pyridone. In some embodiments, the modified nucleobase is selected from 5-substituted pyrimidines, 6-azapyrimidines and N-2, N-6 and O-6 substituted purines, including 2 aminopropyladenine, 5-propynyluracil and 5-propynylcytosine. In some embodiments, the modified nucleobase is a 5-methylcytosine. In some embodiments, at least one nucleoside comprises a cytosine, wherein the cytosine is a 5-methylcytosine. In some embodiments, each cytosine is a 5-methylcytosine. In some embodiments, a 2′-modified nucleoside comprises a 2′-substituent group selected from halo, allyl, amino, azido, —SH, —CN, —OCN, —CF₃, —OCF₃, —O—, —S—, or —N(Rₘ)-alkyl; —O—, —S—, or —N(Rₘ)-alkenyl; —O—, —S— or —N(Rₘ)-alkynyl; —O-alkylenyl-O-alkyl, alkynyl, alkaryl, aralkyl, —O-alkaryl, —O-aralkyl, —O(CH₂)₂SCH₃, —O—(CH₂)₂—O—N(Rₘ)(Rₙ) or —O—CH₂—C(═O)—N(Rₘ)(Rₙ), where each Rₘ and Rₙ is, independently, H, an amino protecting group or substituted or unsubstituted Ci-ioalkyl. These 2′-substituent groups can be further substituted with one or more substituent groups independently selected from hydroxyl, amino, alkoxy, carboxy, benzyl, phenyl, nitro, thiol, thioalkoxy, halogen, alkyl, aryl, alkenyl and alkynyl.

In some embodiments, a 2′-modified nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), —O(CH₂)₂—O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted G_ioalkyl. In some embodiments, a 2′-modified nucleoside comprises a 2′-substituent group selected from F, OCF₃, O—CH₃, OCH₂CH₂OCH₃, 2′—O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In some embodiments, a 2′-modified nucleoside comprises a 2′-substituent group selected from F, O—CH₃, and OCH₂CH₂OCH₃. In some embodiments, a sugar-modified nucleoside is a 4′-thio modified nucleoside. In some embodiments, a sugar-modified nucleoside is a 4′-thio-2′-modified nucleoside. A 4′-thio modified nucleoside has a B-D-ribonucleoside where the 4′-0 replaced with 4′-S. A 4′-thio-2′-modified nucleoside is a 4′-thio modified nucleoside having the 2′-OH replaced with a 2′-substituent group. Suitable 2′-substituent groups include 2′—OCH₃, 2′-O—(CH₂)₂—OCH₃, and 2′-F.

In some embodiments, a modified nucleobase comprises a polycyclic heterocycle. In some embodiments, a modified nucleobase comprises a tricyclic heterocycle. In some embodiments, a modified nucleobase comprises a phenoxazine derivative. In some embodiments, the phenoxazine can be further modified to form a nucleobase known in the art as a G-clamp.

In some embodiments, the oligonucleotide compound comprises a modified oligonucleotide conjugated to one or more moieties which enhance the activity, cellular distribution or cellular uptake of the resulting antisense oligonucleotides. In some such embodiments, the moiety is a cholesterol moiety or a lipid moiety. Additional moieties for conjugation include carbohydrates, phospholipids, biotin, phenazine, folate, phenanthridine, anthraquinone, acridine, fluoresceins, rhodamines, coumarins, and dyes. In some embodiments, a conjugate group is attached directly to a modified oligonucleotide. In some embodiments, a conjugate group is attached to a modified oligonucleotide by a linking moiety selected from amino, hydroxyl, carboxylic acid, thiol, unsaturations (e.g., double or triple bonds), 8-amino-3,6-dioxaoctanoic acid (ADO), succinimidyl4-(N-maleimidomethyl)cyclohexane-1-carboxylate (SMCC), 6-aminohexanoic acid (AHEX or AHA), substituted G-ioalkyl, substituted or unsubstituted C₂-ioalkenyl, and substituted or unsubstituted C₂-ioalkynyl. In some such-embodiments, a substituent group is selected from hydroxyl, amino, alkoxy, carboxy, benzyl, phenyl, nitro, thiol, thioalkoxy, halogen, alkyl, aryl, alkenyl and alkynyl.

In some such embodiments, the oligonucleotide compound comprises a modified oligonucleotide having one or more stabilizing groups that are attached to one or both termini of a modified oligonucleotide to enhance properties such as, for example, nuclease stability. Included in stabilizing groups are cap structures. These terminal modifications protect a modified oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps. Additional cap structures include, but are not limited to, a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threopentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

The present disclosure also provides pharmaceutical compositions comprising one or more of the oligonucleotides described herein. In some embodiments, the oligonucleotide consists of 15 to 40 linked nucleosides, or a salt thereof, wherein the modified oligonucleotide comprises a nucleobase sequence that is at least 80% identical to the nucleobase sequence of any one of SEQ ID NOs:1, 2 or 3 as described in detail above and a pharmaceutically acceptable carrier or diluent. In each of these embodiments, the oligonucleotide can be a modified oligonucleotide.

In some embodiments, the compositions may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions, such as dyes, flavoring agents, preservatives, antioxidants, opacifiers, thickening agents and stabilizers. However, such materials, when added, should not unduly interfere with the biological activities of the components of the compositions. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation. In some embodiments, pharmaceutical compositions comprise one or more modified oligonucleotides and one or more excipients. In some such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone. In some embodiments, a pharmaceutical composition is prepared using known techniques, including, but not limited to mixing, dissolving, granulating, dragee-making, levigating, emulsifying, encapsulating, entrapping or tab letting processes.

In some embodiments, a pharmaceutical composition is a liquid (e.g., a suspension, elixir and/or solution). In some such embodiments, a liquid pharmaceutical composition is prepared using ingredients known in the art, including, but not limited to, water, glycols, oils, alcohols, flavoring agents, preservatives, and coloring agents.

In some embodiments, a pharmaceutical composition is a solid (e.g., a powder, tablet, and/or capsule). In some such embodiments, a solid pharmaceutical composition comprising one or more oligonucleotides is prepared using ingredients known in the art, including, but not limited to, starches, sugars, diluents, granulating agents, lubricants, binders, and disintegrating agents.

In some embodiments, a pharmaceutical composition is formulated as a depot preparation. Some such depot preparations are typically longer acting than non-depot preparations. In some embodiments, such preparations are administered by implantation (for example subcutaneously or intramuscularly) or by intramuscular injection. In some embodiments, depot preparations are prepared using suitable polymeric or hydrophobic materials (for example an emulsion in an acceptable oil) or ion exchange resins, or as sparingly soluble derivatives, for example, as a sparingly soluble salt.

In some embodiments, a pharmaceutical composition comprises a delivery system. Examples of delivery systems include, but are not limited to, liposomes and emulsions. Delivery systems are useful for preparing pharmaceutical compositions including those comprising hydrophobic compounds. In some embodiments, some organic solvents such as dimethylsulfoxide are used. In some embodiments, presently available RNAi packaging technology can be used to packing the miRNA in lipid complexes and to deliver the miRNA. The delivery system can also comprise nanoparticles or nano-complexes. The delivery system can also comprise bacterial mini-cells comprising RNA duplexes.

In some embodiments, a pharmaceutical composition comprises one or more tissue-specific delivery molecules designed to deliver the one or more pharmaceutical agents to specific tissues or cell types. For example, in some embodiments, pharmaceutical compositions include liposomes coated with a tissue-specific antibody.

In some embodiments, a pharmaceutical composition comprises a cosolvent system. Some such co-solvent systems comprise, for example, benzyl alcohol, a nonpolar surfactant, a water-miscible organic polymer, and an aqueous phase. In some embodiments, such cosolvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycoBOO. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In some embodiments, a pharmaceutical composition comprises a sustained-release system. A non-limiting example of such a sustained-release system is a semi-permeable matrix of solid hydrophobic polymers. In some embodiments, sustained-release systems may, depending on their chemical nature, release pharmaceutical agents over a period of hours, days, weeks or months.

In some embodiments, a pharmaceutical composition is prepared for oral administration. In some such embodiments, a pharmaceutical composition is formulated by combining one or more compounds comprising any one or more of the oligonucleotides described herein with one or more pharmaceutically acceptable carriers. Some such carriers enable pharmaceutical compositions to be formulated as tablets, pills, dragees, capsules, liquids, gels, syrups, slurries, suspensions and the like, for oral ingestion by a subject. In some embodiments, pharmaceutical compositions for oral use are obtained by mixing oligonucleotide and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In some embodiments, such a mixture is optionally ground and auxiliaries are optionally added. In some embodiments, pharmaceutical compositions are formed to obtain tablets or dragee cores. In some embodiments, disintegrating agents (e.g., cross-linked polyvinylpyrrolidone, agar, or alginic acid or a salt thereof, such as sodium alginate) are added. In some embodiments, dragee cores are provided with coatings. In some such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In some embodiments, pharmaceutical compositions for oral administration are push-fit capsules made of gelatin. Some such push-fit capsules comprise one or more of the oligonucleotides described herein in admixture with one or more filler such as lactose, binders such as starches, and/or lubricants such as talc or magnesium stearate and, optionally, stabilizers. In some embodiments, pharmaceutical compositions for oral administration are soft, sealed capsules made of gelatin and a plasticizer, such as glycerol or sorbitol. In some soft capsules, one or more of the oligonucleotides described herein are be dissolved or suspended in suitable liquids, such as fatty oils, liquid paraffin, or liquid polyethylene glycols. In addition, stabilizers may be added.

In some embodiments, pharmaceutical compositions are prepared for buccal administration. Some such pharmaceutical compositions are tablets or lozenges formulated in conventional manner.

In some embodiments, a pharmaceutical composition is prepared for administration by injection (e.g., intravenous, intraperitoneal, subcutaneous, intramuscular, etc.). In some such embodiments, a pharmaceutical composition comprises a carrier and is formulated in aqueous solution, such as water or physiologically compatible buffers such as Hanks's solution, Ringer's solution, or physiological saline buffer. In some embodiments, other ingredients are included (e.g., ingredients that aid in solubility or serve as preservatives). In some embodiments, injectable suspensions are prepared using appropriate liquid carriers, suspending agents and the like. Some pharmaceutical compositions for injection are presented in unit dosage form, e.g., in ampoules or in multi-dose containers. Some pharmaceutical compositions for injection are suspensions, solutions or emulsions in oily or aqueous vehicles, and may contain formulatory agents such as suspending, stabilizing and/or dispersing agents. Some solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the oligonucleotides described herein to allow for the preparation of highly concentrated solutions.

In some embodiments, a pharmaceutical composition is prepared for transmucosal administration. In some such embodiments penetrants appropriate to the barrier to be permeated are used in the formulation. Such penetrants are generally known in the art.

In some embodiments, a pharmaceutical composition is prepared for administration by inhalation. Some such pharmaceutical compositions for inhalation are prepared in the form of an aerosol spray in a pressurized pack or a nebulizer. Some such pharmaceutical compositions comprise a propellant, e.g., dichlorodifluoromethane, trichlorofluoromethane, dichlorotetrafluoroethane, carbon dioxide or other suitable gas. In some embodiments using a pressurized aerosol, the dosage unit may be determined with a valve that delivers a metered amount. In some embodiments, capsules and cartridges for use in an inhaler or insufflator may be formulated. Some such formulations comprise a powder mixture of one or more of the oligonucleotides described herein and a suitable powder base such as lactose or starch. In some embodiments, a pharmaceutical composition is prepared for rectal administration, such as a suppositories or retention enema. Some such pharmaceutical compositions comprise known ingredients, such as cocoa butter and/or other glycerides.

In some embodiments, a pharmaceutical composition is prepared for topical administration. Some such pharmaceutical compositions comprise bland moisturizing bases, such as ointments, creams, gels, liniments, lotions, and salves. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In some embodiments, a pharmaceutical composition comprises a modified oligonucleotide in a therapeutically effective amount. In some embodiments, the therapeutically effective amount is sufficient to prevent, alleviate or ameliorate symptoms of a disease or to prolong the survival of the subject being treated. Determination of a therapeutically effective amount is well within the capability of those skilled in the art. In some embodiments, the pharmaceutical composition may further comprise at least one additional therapeutic agent.

The present invention provides methods of treating an inflammatory disease comprising administering to a subject in need thereof one or more of the oligonucleotides described herein, and/or a pharmaceutical agent that induces the production of the one or more oligonucleotides. In some embodiments, the oligonucleotide consists of 15 to 40 linked nucleosides, wherein the oligonucleotide comprises a nucleobase sequence that is at least 80% identical to the nucleobase sequence of any one of SEQ ID NOs:1, 2 or 3. In some embodiments, the oligonucleotide is a modified oligonucleotide as described herein. In some embodiments, the inflammatory disease is osteoarthritis or rheumatoid arthritis. In some embodiments, the subject has been diagnosed with osteoarthritis. In some embodiments, the subject has been diagnosed with rheumatoid arthritis. In some embodiments, the inflammatory disease is Bechterew's disease, psoriatic arthritis, arthritis associated with inflammatory bowel disease, and other and HLA-related autoimmune diseases like multiple sclerosis (MS), and Systemic lupus erythematosus (SLE). In still other embodiments, the inflammatory disease in an inflammatory skin disease such as psoriasis, dermatitis (eczema), rosacea, and seborrheic dermatitis.

In some embodiments, the methods described herein use one or more oligonucleotides or modified oligonucleotides that is/are modified versions of the 5′ isomiR of any one of SEQ ID NOs: 1, 2 or 3. The oligonucleotides or modified oligonucleotides can be administered with or without being integrated into a vector. The oligonucleotides or modified oligonucleotides can also be used in the form of double stranded entities, whereby the appropriate strand is produced inside a cell.

In some embodiments, administration of an oligonucleotide comprises intra-articular administration, intravenous administration, subcutaneous administration, transdermal administration, intraperitoneal administration. In some particularly preferred embodiments, administration of an oligonucleotide comprises intra-articular administration.

In some embodiments, any one or more of the oligonucleotides described herein is administered at a dose selected from 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, and 800 mg. The oligonucleotide may be administered one per day, once per week, once per two weeks, once per three weeks, or once per four weeks.

In some preferred embodiments, the administration of an oligonucleotide of the present invention results in relief or amelioration of one or more symptoms of osteoarthritis. Symptoms of osteoarthritis that are relieved or ameliorated include pain or aching, joint stiffness, decreased range of motion, swelling and inflammation.

In some preferred embodiments, the administration of an oligonucleotide of the present invention results in relief or amelioration of one or more symptoms of rheumatoid arthritis. Symptoms of rheumatoid arthritis that are relieved or ameliorated include pain or aching, joint tenderness, joint stiffness, decreased range of motion, inflammation, fatigue and fever. In some preferred embodiments, the administration of an oligonucleotide of the present invention results in down-regulation of the interferon gamma cascade in the tissue of a subject. In some preferred embodiments, the administration of an oligonucleotide of the present invention results in down-regulation of the interferon gamma cascade in the tissue of a subject at the site of administration. In some preferred embodiments, the administration of an oligonucleotide of the present invention results in down-regulation of HLA class II genes in a joint or tissue of a subject. In some preferred embodiments, the administration of an oligonucleotide of the present invention results in down-regulation of HLA class II genes in a joint or tissue of a subject at the site of administration. In some preferred embodiments, the administration of an oligonucleotide of the present invention results in down-regulation of HLA class I genes in a joint or tissue of a subject. In some preferred embodiments, the administration of an oligonucleotide of the present invention results in down-regulation of HLA class I genes in a joint or tissue of a subject at the site of administration. In some embodiments, the tissue is cartilage.

In some embodiments, such pharmaceutical compositions comprise any one or more of the oligonucleotides or modified oligonucleotides described herein in a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg 125 mg 130 mg 135 mg 140 mg, 145 mg, 150 mg, 155 mg, 160 mg, 165 mg, 170 mg, 175 mg 180 mg 185 mg 190 mg 195 mg, 200 mg, 205 mg, 210 mg, 215 mg, 220 mg, 225 mg, 230 mg 235 mg 240 mg 245 mg, 250 mg, 255 mg, 260 mg, 265 mg, 270 mg, 270 mg, 280 mg, 285 mg 290 mg 295 mg 300 mg 305 mg, 310 mg, 315 mg, 320 mg, 325 mg, 330 mg, 335 mg, 340 mg 345 mg, 350 mg 355 mg 360 mg, 365 mg, 370 mg, 375 mg, 380 mg, 385 mg, 390 mg, 395 mg 400 mg 405 mg 410 mg 415 mg, 420 mg, 425 mg, 430 mg, 435 mg, 440 mg, 445 mg, 450 mg 455 mg, 460 mg 465 mg 470 mg, 475 mg, 480 mg, 485 mg, 490 mg, 495 mg, 500 mg, 505 mg 510 mg 515 mg 520 mg 525 mg, 530 mg, 535 mg, 540 mg, 545 mg, 550 mg, 555 mg, 560 mg 565 mg 570 mg 575 mg 580 mg, 585 mg, 590 mg, 595 mg, 600 mg, 605 mg, 610 mg, 615 mg 620 mg 625 mg 630 mg 635 mg, 640 mg, 645 mg, 650 mg, 655 mg, 660 mg, 665 mg, 670 mg 675 mg 680 mg 685 mg 690 mg, 695 mg, 700 mg, 705 mg, 710 mg, 715 mg, 720 mg, 725 mg 730 mg 735 mg 740 mg 745 mg, 750 mg, 755 mg, 760 mg, 765 mg, 770 mg, 775 mg, 780 mg 785 mg 790 mg 795 mg and 800 mg. In some such embodiments, a pharmaceutical composition comprises a dose of modified oligonucleotide selected from 25 mg, 50 mg, 75 mg, 100 mg, 150 mg, 200 mg, 250 mg, 300 mg, 350 mg, 400 mg, 500 mg, 600 mg, 700 mg, and 800 mg.

In some embodiments, a pharmaceutical agent is sterile lyophilized oligonucleotide that is reconstituted with a suitable diluent, e.g., sterile water for injection or sterile saline for injection. The reconstituted product is administered as a subcutaneous injection or as an intravenous infusion after dilution into saline. The lyophilized drug product consists of any one or more of the oligonucleotides or modified oligonucleotides described herein which has been prepared in water for injection, or in saline for injection, adjusted to pH 7.0-9.0 with acid or base during preparation, and then lyophilized. The lyophilized modified oligonucleotide may be 25-800 mg of any one or more of the oligonucleotides or modified oligonucleotides described herein. It is understood that this encompasses 25, 50, 75, 100, 125, 150, 175, 200, 225, 250, 275, 300, 325, 350, 375, 425, 450,475, 500, 525, 550,575, 600, 625, 650, 675, 700, 725, 750, 775, and 800 mg of modified lyophilized oligonucleotide. The lyophilized drug product may be packaged in a 2 mL Type I, clear glass vial (ammonium sulfate-treated), stoppered with a bromobutyl rubber closure and sealed with an aluminum FLIP-OFF® overseal.

The present disclosure also provides any one or more of the oligonucleotide compounds described herein, or compositions comprising the same, for use in the manufacture of a medicament for treating or preventing an inflammatory disease, preferably but not limited to osteoarthritis, rheumatoid arthritis, Bechterew's disease, psoriatic arthritis, arthritis associated with inflammatory bowel disease, and other and HLA-related autoimmune diseases like multiple sclerosis (MS), Systemic lupus erythematosus (SLE) and inflammatory skin diseases such as psoriasis, dermatitis (eczema), rosacea, and seborrheic dermatitis.

The present disclosure also provides uses of any one or more of the oligonucleotide compounds described herein, or compositions comprising the same, for treating or preventing an inflammatory disease, preferably but not limited to osteoarthritis, rheumatoid arthritis Bechterew's disease, psoriatic arthritis, arthritis associated with inflammatory bowel disease, and other and HLA-related autoimmune diseases like multiple sclerosis (MS), Systemic lupus erythematosus (SLE) and inflammatory skin diseases such as psoriasis, dermatitis (eczema), rosacea, and seborrheic dermatitis.

The present disclosure also provides uses of any one or more of the oligonucleotide compounds described herein, or compositions comprising the same, in the manufacture of a medicament for treating or preventing osteoarthritis.

The present disclosure also provides any one or more of the oligonucleotide compounds described herein, or compositions comprising the same, or methods of preparing the same, or methods of using the same, or uses any one or more of the oligonucleotide compounds described herein, or compositions comprising the same, substantially as described with reference to the accompanying examples and/or figures.

In order that the subject matter disclosed herein may be more efficiently understood, examples are provided below. It should be understood that these examples are for illustrative purposes only and are not to be construed as limiting the claimed subject matter in any manner.

### EXPERIMENTAL

### Example 1

MicroRNA-140-3p is the most prevalent form of canonical miR-140 in native chondrocytes. IsomiRs are sequence variants of microRNAs with potentially distinct functionalities. Here we present functional studies of canonical microRNA-140-3p and two of its most prevalent isomiRs, a 5′ isomiR and a 3′ isomiR, in an inflammation-induced model of osteoarthritis (OA).

Canonical miR-140-3p, the 5′ isomiR and the 3′ isomiR were overexpressed separately in chondrocytes from three donors and subsequently subjected to an inflammatory milieu mediated by interleukin 1 beta and tumor necrosis factor alpha. RNA sequencing was performed on the cells to investigate the altered transcriptomes, RT-qPCR was performed to validate important observations, and western blot analysis was carried out to further study key inflammatory molecules.

The three microRNAs downregulated many of the same genes. However, the 5′ isomiR showed a much greater target spectrum compared to the other two miRNAs, and downregulated cascades of genes downstream of interferon beta, interferon gamma and interleukin 1 beta as well as genes involved in several other inflammatory and antiviral pathways. In addition the 5′ isomiR downregulated practically all HLA class II and class I genes.

Introduction of the 5′ isomiR led to downregulation of genes essential for some of the most important inflammation cascades and virtual silencing of genes responsible for antigen presentation. These observations may indicate a very promising therapeutic potential for the 5′ isomiR for OA and several inflammatory conditions, particularly HLA associated immune conditions including many arthritic diseases.

### Methods and Materials

Isolation and culture of human articular chondrocytes (ACs). ACs were isolated from discarded OA cartilage tissue after total knee replacement surgery and cultured as previously described. Only tissue with no macroscopic signs of OA was used. All donors provided written, informed consent. The study was approved by the Regional Committee for Medical Research Ethics, Southern Norway. Briefly, the cartilage was cut into tiny pieces and subsequently digested with Collagenase type XI (Sigma-Aldrich, St. Louis, MO) at 37° C. for 90-120 min. Chondrocytes were washed three times and resuspended in culture medium consisting of Dulbecco's modified Eagle's medium/F12 (Gibco/ThermoFisher Scientific, Waltham, MA, USA) supplemented with 10% human plasma (Octaplasma AB, Oslo Blood Bank, Norway) supplemented with platelet lysate (corresponding to 10⁹ platelets/ml plasma) (PLP), 100 units/mL penicillin, 100 μg/mL streptomycin, and 2.5 μg/mL amphotericin B22. PLP was prepared as previously described. The culture medium was changed every 3-4 days.

After the first passage amphotericin B was removed. At 70-80% confluence, cells were detached with trypsin-EDTA (Sigma-Aldrich) and seeded into new culture flasks. MicroRNA mimics, transfection and stimulation with IL1β and TNFα. The Amaxa nucleofector system and the Amaxa Human Chondrocyte Nucleofector Kit were used for electroporation following the protocols from the manufacturer (Lonza, Walkersville, MD). Briefly, each reaction contained 1.0×10⁶ cells, 5 μM of miRvana mimics in a total volume of 100 μl nucleofection solution. The cells were seeded in 20% PLP without antibiotics and left to recover overnight. The following day (day 1) the medium was changed to 10% PLP with 1% penicillin/streptomycin. On day 4 ACs were stimulated with 0.1 ng/ml recombinant IL1β (IL1β) and 10 ng/ml TNFα (R&D Systems, Minneapolis, MN) for 24 hours before harvesting for analysis.

Isolation of miRNA, cDNA synthesis, and RT-qPCR. Total RNA containing miRNAs was isolated using the miRNeasy mini kit according to manufacturer's protocol (Qiagen, Germantown, MD). cDNA synthesis and RT-qPCR were performed following protocols from the manufacturer using the Taqman High capacity cDNA Reverse Transcription Kit for mRNA and Taqman MicroRNA Reverse Transcription Kit for microRNAs (Thermo Fisher Scientific, Waltham, MA, USA). 2 ng miRNA in a total volume of 15 μl, and for other genes 200 ng RNA in a total volume of 15 μl was reverse transcribed into cDNA. All samples were run in technical triplicates. Each replicate contained 1.33 μl cDNA in a total volume of 15 μl for miRNAs and 0.2 μl cDNA in a total volume of 15 μl for mRNAs. The thermocycling parameters were 95° C. for 10 minutes followed by 40 cycles of 95° C. for 15 seconds and 60° C. for 1 minute. U6 was used as endogenous control for miRNAs and GAPDH was used as endogenous control for mRNAs. RT-qPCR results are shown as relative fold changes using mean values from technical triplicates with a 95% confidence interval. All donors are shown separately in the figures.

Western blotting. Cell lysates corresponding to 200,000 cells were loaded onto a 4-20% gradient or 10% polyacrylamide gel (Bio-Rad, Hercules, CA). Proteins were separated by gel electrophoresis, transferred to PVDF membranes and incubated with appropriate antibodies before visualizing the bands using the myECL imager (Thermo Fisher Scientific).

RNA-Sequencing. Sequence libraries from mRNA was prepared using the TruSeq Stranded mRNA kit (Illumina) at the Norwegian Sequencing Center, Oslo University Hospital, Ullevål. BBMap v34.56 was used to remove low quality reads and adapter sequences. HiSat2 v2.1.0 for mapping reads to the genome 139 and Samtools v1.2 to convert SAM files to BAM files. The BAM files was uploaded and analysed using the Seqmonk software. The DEseq² package in Seqmonk was used for differential expression analysis. ShinyGO v0.61 was used for GO-term and Gene set enrichment analysis and for in silico prediction of transcription factor-binding sites.

### Results

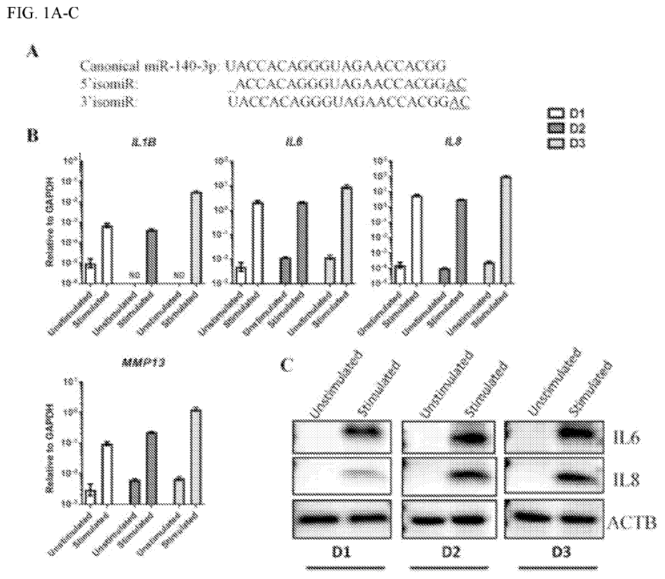

The sequences of the canonical miR-140-3p and our chosen 5′ and 3′ isomiR are shown in FIG. 1A. The 5′ isomiR was chosen because it was the sequence with the highest expression in articular chondrocytes in the study performed by Haseeb et. al and it was also prevalent in our recent study of miRNAs and isomiRs in plasma extracellular vesicles both in patients with OA and controls31,32. It lacks the first nt of the canonical miR-140-3p, and has two additional nt at the 3′ end, A and C. The 3′ isomiR was chosen because it has the same two additional nt at the 3′ end, and is otherwise identical to the canonical sequence. The 3′ isomiR, too, is prevalent in the Haseeb et al. study and in our own isomiR analyses. An inflammation-induced model of OA was established in vitro by stimulating cultured articular chondrocytes with IL1β and TNFα. IL1B and TNFα strongly upregulated the mRNA expression of the OA-inducing cytokines IL1B, IL6, IL8 and the matrix degrading enzyme MMP13 in cells from all donors (FIG. 1B). FIG. 1C shows induced protein levels of OA associated cytokines IL6 and IL8 in response to stimulation by IL1β and TNFα.

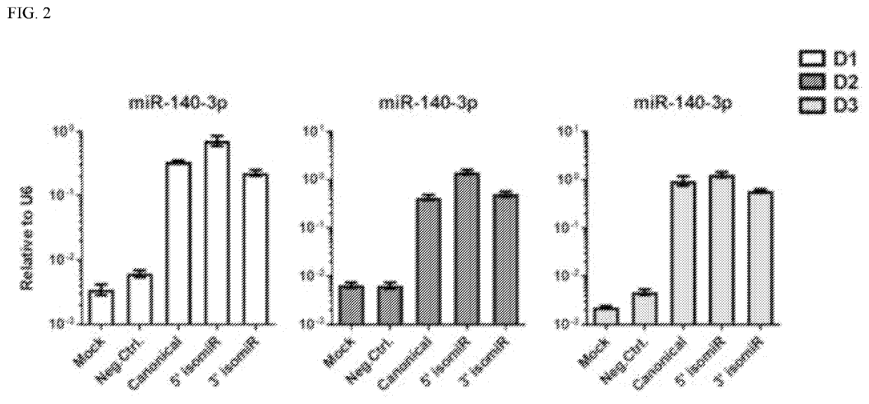

Our previous study showed that canonical miR-140-3p inhibited IL1β and TNFα-mediated inflammation. To investigate the functional role of the 5′ isomiR and 3′ isomiR in OA compared to their canonical sequence, we transfected chondrocytes with each of the miRNAs 161 separately. Higher levels of the miRNA sequences were confirmed in all three donors (FIG. 2). Mock (electroporated cells) and transfection of a negative control sequence were both used as controls. The Taqman MicroRNA assay used for RT-qPCR is designed for the canonical miR-140-3p sequence, but it amplified 164 both isomiRs as well. This was tested for all three sequences by RT-qPCR amplification directly from the reagent tube (data not shown).

miR-140-3p and its isomiRs have Different Effects on Key Inflammatory Cytokines

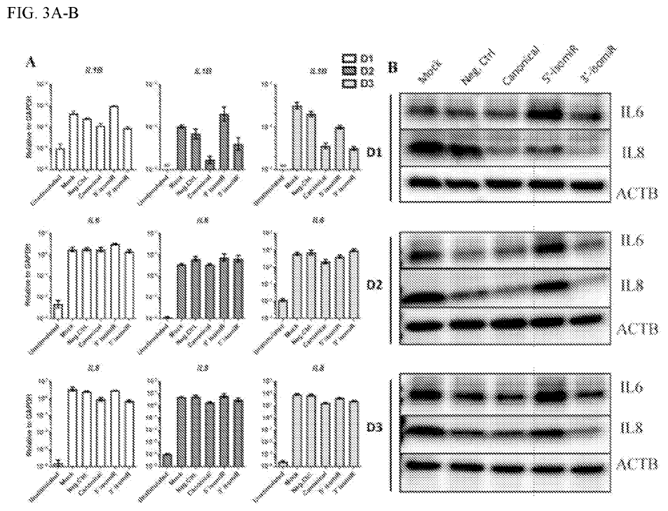

We then wanted to study the effect of the different miRNAs on key inflammatory cytokines within the inflammatory milieu mediated by IL1β and TNFα. Canonical miR-140-3p and its 3′ isomiR downregulated IL1B and, marginally, IL8 (FIG. 3A). The 5′ isomiR upregulated IL6 and IL8 protein compared with canonical miR-140-3p and the 3′ isomiR, and practically always also compared with the negative control. At the protein level (FIG. 3B), one consistent observation was that IL6 and IL8 were both downregulated by the negative control relative to mock.

RNA Sequencing Revealed Both Unique and Overlapping Changes in Gene Expression Following Transfection of the Canonical miR-140-3p or its isomiRs

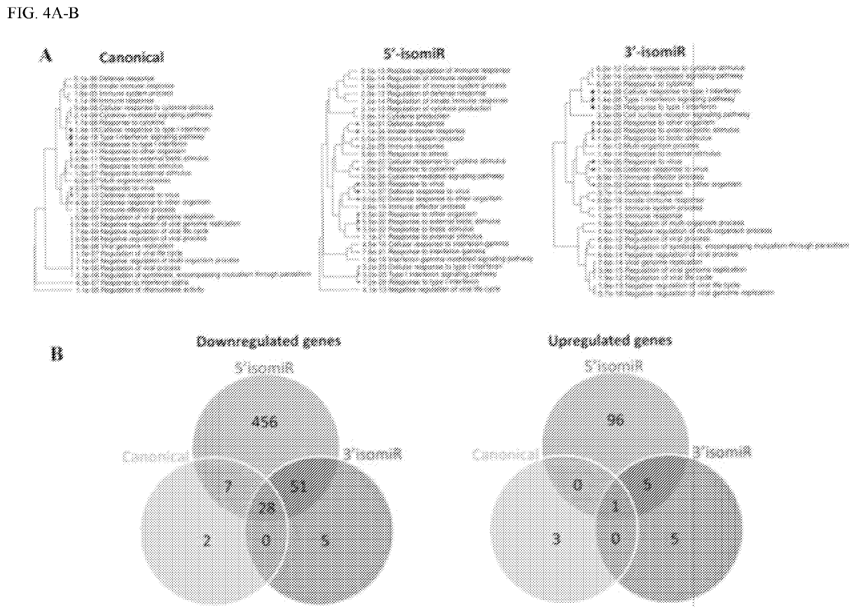

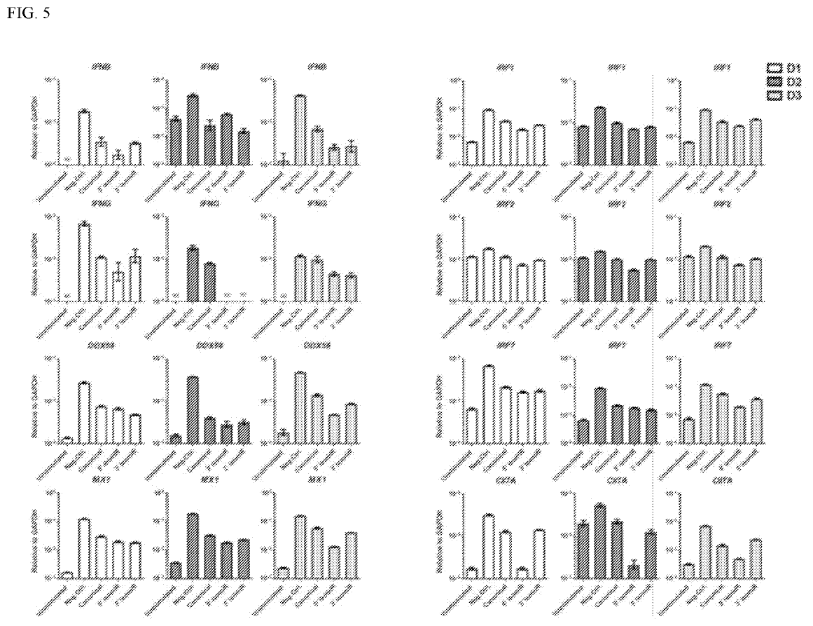

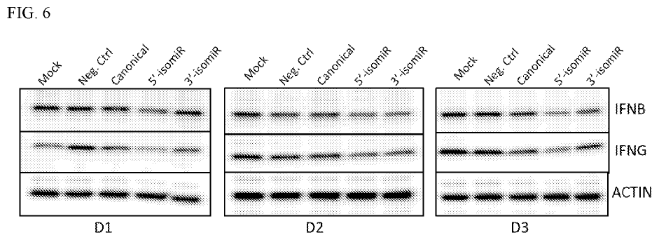

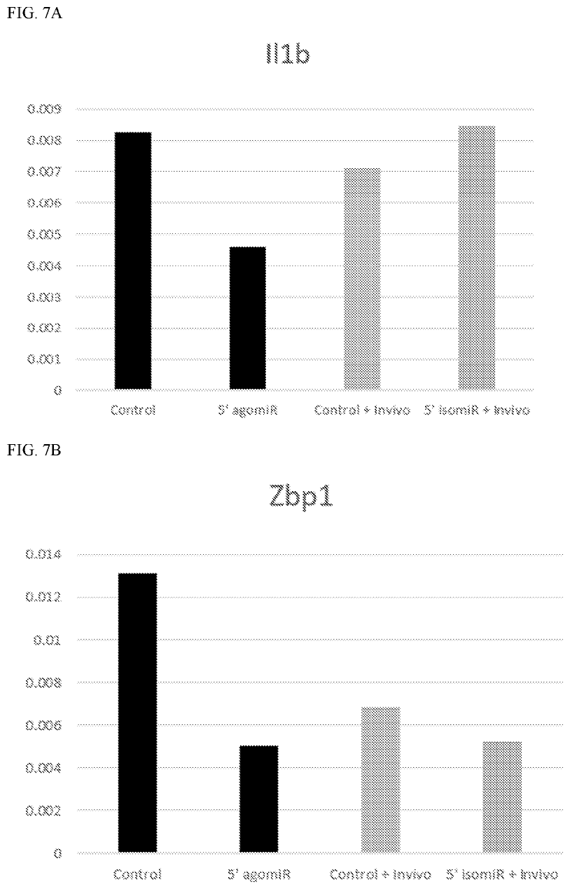

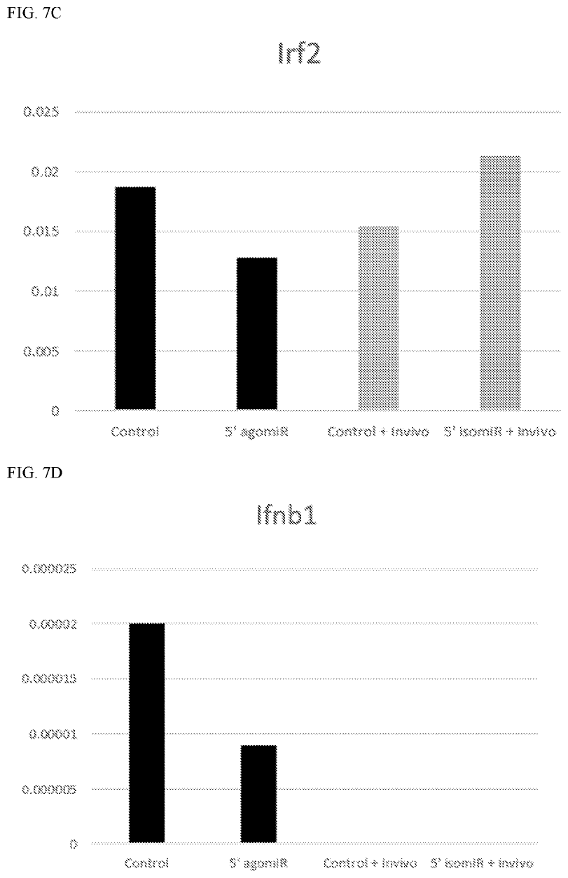

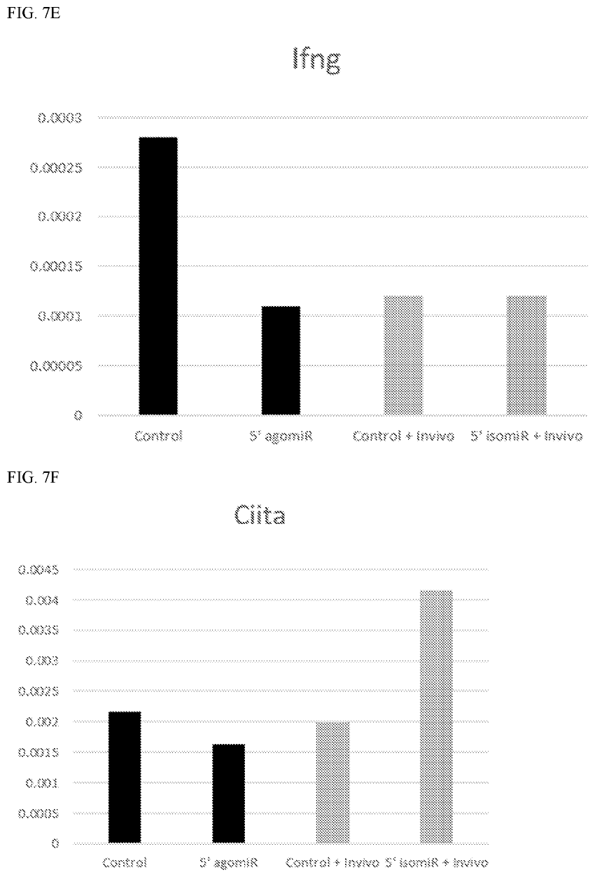

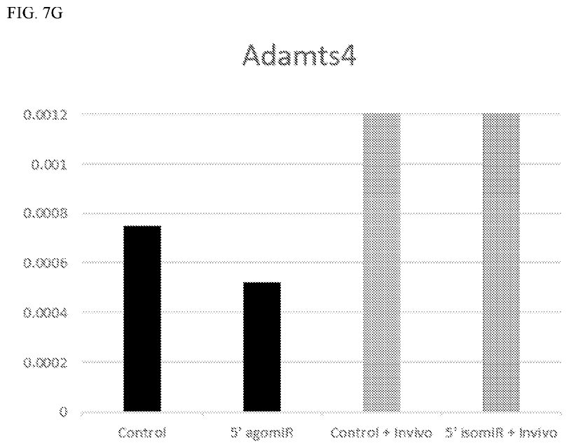

In order to further unravel the biological impact of miR-140-3p and the two isomiRs we performed RNA sequencing on the same cells as were used to produce the results described in FIG. 3. The negative control sequence was used as control, as there were not significantly differentially expressed genes compared with mock control. Canonical miR-140-3p, 5′ isomiR and 3′ isomiR downregulated the expression of 37, 542, and 84 genes and upregulated the expression of 4, 102, and 11 genes, respectively (FDR<0.05, cut-off at 2 fold difference, data not shown). Gene Ontology (GO)-analysis of downregulated genes showed 184 that all three miRNAs regulated the same or related biological processes (FIG. 4A).

Almost all GO-terms were related to immune responses such as type I interferon responses, innate immunity response, defense responses virus and to other pathogens, suggesting that many of the same genes or gene families were targeted by the three miRNAs. And this was, indeed, the case. Having the same seed sequence 188 as the canonical miR-140-3p, the 3′ isomir would be expected to have a similar spectrum of target mRNAs. We found that 28 of the 37 mRNAs (76%) downregulated by the canonical miR-140-3p were also downregulated by the 3′ isomiR (FIG. 4B). The 3′ isomiR also downregulated another 56 mRNAs, of which 51 (91%) were also downregulated by the 5′ isomiR. The 28 mRNAs that were downregulated by both the canonical and the 3′ isomiR were also downregulated by the 5′ isomiR. This left very few mRNAs, two and five, to be uniquely downregulated by the canonical miR-140-3p and its 3′ isomiR, respectively (FIG. 4B). Thus, most of the following results will be related to the downregulatory effect of the 5′ isomiR.

The 5′ isomiR Downregulated Cascades of Immunologically Active mRNAs

The miRDB database predicted 100 genes to be targeted by both canonical miR-140-3p and 5′ isomiR (data not shown) suggesting several common targets despite having different seeds. Among the 542 genes downregulated by the 5′ isomiR, the database predicted 27 to be targets of the 5′isomiR (data not shown). Each of these could, potentially, initiate cascades of downstream events leading to further downregulation of mRNAs. Scrutiny of the 27 predicted targets downregulated by the 5′ isomiR revealed three possible cascade initiating candidates: IRF2, RNASE4 and TNERSF14, (data not shown).

Examining the list of GO terms for evidence of cascades of downregulated mRNAs following 5′ isomiR transfection, the type I interferon (IFNA/IFNB) and interferon gamma (IFNG) pathways turned up as highly significant. Neither IFNA nor IFNB were detected to be significantly downregulated. However, RT-qPCR analysis showed that IFNB was in fact downregulated (FIG. 5), while IFNA was not detected. IFNG was downregulated by 32-fold (validated by PCR in FIG. 5). IL12A and IL12RB1 are involved in the induction of IFNG transcription and were downregulated (data not shown). CASP1 is required for the conversion of pro-212 ILβ1 to IL1β33 and was also downregulated (data not shown). Another downregulated mRNA that is involved in IL1β signaling was MYD88 (data not shown). Together this may explain the downregulation of molecules of the IL1β cascade.

Another cascade initiator downregulated by the 5′ isomiR is the cytoplasmic sensor of viral nucleic acids DDX58 (also known as RIG-1), which activates a cascade of antiviral responses (validated in FIG. 5). MX1 is another molecule that is involved in cellular anti-viral response and was also downregulated (See FIG. 5). Yet others are the transcription factors IRF1, IRF2 and IRF7 (See FIG. 5), and several cytokines.

The data collected identified another interesting series of downregulated genes: practically all genes and pseudogenes in the HLA class II histocompatibility antigens region and, to a lesser extent also the HLA class I region on chromosome 6 were downregulated: HLA-DMA, HLA-DOB, HLA-DPA1, HLA-DPB1, HLA-DQA1, HLA-DOB1, HLA-DRB1, HLA-DRB5 as well as HLA class I genes HLA-A, HLA-B, HLA-F, HLA-K, but not the HLA class III region genes. In addition TAP1 mRNA, encoding a protein which is essential for the availability of cytosolic peptides to HLA class I molecules in the endoplasmic reticulum, was also downregulated. The master regulator of the HLA class II region, Class II transactivator CIITA, is induced by IFNG34. To our surprise, CIITA was not downregulated by the 5′ isomiR according to our sequencing analysis. However, by RT-qPCR CIITA was found to be greatly downregulated to, or below, the level found in unstimulated chondrocytes (FIG. 5).

Downregulation of CIITA, then, most likely explains the downregulation of the HLA class II mRNAs. Downregulated genes IRF/35 and AIM236 may also regulate HLA class II expression as both act through CIITA. For the HLA class I region the explanation probably lies in the downregulation of the master regulator, NLRC5 mRNA (data not shown)³⁷. Although the majority of the genes downregulated by 5′ isomiR are positive regulators of inflammation and immune responses, two genes have been shown to be negative regulators of IL6, SOCS338,39 and ZC3H12D40. The downregulation of these two genes could perhaps explain why 5′ isomiR leads to upregulation of IL6 and IL8. Interestingly, the promotors of the downregulated genes were enriched for DNA binding sites for several immunoregulatory transcription factors, including IRF7, IRF1 and IRF2 that were also downregulated by all three miRNAs (See FIG. 5). Thus downregulation of these transcription factors most likely contributes to downregulation of many of the genes. In addition the cartilage and matrix associated genes ACAN, HAPLN3, PRG4, FMOD, PRELP, CCN6/WISP3, ROR2, CEMIP, ADAMTS4, MMP1 and MMP12 were also downregulated by 5′ isomiR. 5′ isomiR seemed to also target many non-coding genes that are either pseudogenes, non-coding or long non-coding RNA. 18.5% of the downregulated genes were non-protein coding genes.

Upregulated mRNAs

Only four genes were upregulated by canonical miR-140-3p and 11 genes were upregulated by the 3′ isomiR (data not shown). 5′ isomiR transfection led to upregulation of 102 mRNAs, some of which are integrins, growth factors and matrix related enzymes (data not shown). One gene was upregulated by all three miRNAs, while 45% (5 of 11) genes upregulated by 3′ isomiR were also upregulated by 5′ isomiR (FIG. 4B).

### Discussion

miRNAs are potent regulatory molecules with interesting therapeutical potential for OA and other diseases. Their repertoire is only increasing in complexity with the emergence of deep sequencing data revealing numerous isomiRs of canonical miRNAs. In order to unleash that therapeutical potential more knowledge is required to understand how isomiRs operate together with or in comparison to their canonical sequences. This study aimed to unravel the functionality of the most prevalent cartilage miRNAs, miR-140-3p, and two of its isomiRs, in an inflammation-induced in vitro model of OA.

The 5′ isomiR downregulated by far the most mRNAs and a great majority of the downregulated genes were components of functionally interacting cascades, where the downregulation of one gene by the 5′ isomiR most likely led to the downregulation of a number of mRNAs in the same pathway. One such cascade is induced by IFNγ. IFNG is not constitutively expressed in chondrocytes as far as we know, and our RT-qPCR data support this claim. Also IL1β and TNFα are not known to induce the expression of IFNG, but these cytokines are shown here to do so. The synthesis of IFNγ as a consequence of IL1β and TNFα exposure will induce the expression of a cascade of molecules. In cells transfected with the 5′ isomiR IFNG was considerably downregulated, and RT-qPCR showed that the 3′ isomiR and canonical miR-140-3p also downregulated IFNG, albeit to a lesser extent. IFNG is not known to be a direct target of the 5′ isomiR. However, IL12A and IL12RB1 are involved in the induction of IFNG transcription, and their mRNAs are both downregulated suggesting that they, in part, may be responsible for the downregulation of IFNG mRNA following transfection of the 5′ isomiR. The IFNγ and IFNα/β signaling pathways are known to cross talk at multiple levels, suggesting that downregulation of IFNG may reduce the level of mRNAs that are also classified to the IFNα/β pathway. Moreover RT-qPCR analysis showed that IFNB was also downregulated, which could affect several genes in the IFNα/β pathway.

Other cascade events are probably initiated by the downregulation of functional IL1β, other cytokines, IRF's 1, 2 and 7 and DDX58. Together these cascades make up inflammasomes generated by many different stimuli, and their downregulation may turn out to have very interesting therapeutic potential. Downregulation of these cascades probably account for the vast majority of downregulated mRNAs in this model system.

Another immunologically important effect of the overexpression of the 5′ isomiR is the downregulation of practically the entire HLA class II, as well as the HLA class I region on the short arm of chromosome 6 and TAP1. HLA class II and class I regions are transcriptionally regulated by master regulators and members of the NOD-like receptor family CIITA42 and NLRC537 (CITA), respectively. Both of these are induced by IFNγ, and both were shown to be downregulated by the 5′ isomiR, perhaps as a consequence of the downregulation of IFNG. The downregulated genes IRF1 and AIM2 may also regulate HLA class II expression. Both genes are also induced by IFNγ, and act through CIITA. A molecule that inhibits the presentation of HLA class II and class I antigens may well turn out to have therapeutic potential, for instance in cases of HLA restricted autoimmune disease including many arthritic diseases.

The validation experiments with RT-qPCR mostly revealed close similarity between data obtained by RNA-seq and RT-qPCR. However, stringent statistical analysis might disqualify a gene from significance; for instance DDX58 was significantly downregulated by 5′isomiR and 3′isomiR, but not by canonical miR-140-3p in the RNA-seq data, while RT-qPCR validated its downregulation by the canonical sequence. CIITA and IFNB were not detected by RNA-seq, however RT-qPCR showed their downregulation by all three miRNAs. These validation experiments also suggest that there are probably more genes regulated in common by all three miRNAs than what the RNA-seq data showed.

A recent study investigated miR-140-3p and two of its isomiRs, termed miR-140-3p.1 and miR-140-3p.2, in chondrocytes in a non 310-inflammatory system. It showed that overexpression of miR-140-3p.1, the same isomiR we termed 5′ isomiR in this disclosure, led to the downregulation of 693 genes. Of these, 104 of them were also downregulated in our system. Those 104 genes are involved in innate immune response, viral response and type I interferon signaling pathways.

The 5′ isomiR also downregulated several cartilage and matrix related genes such as PRELP44, FMOD45, CCN6/WISP3 and ROR2. In addition ACAN, the major component of cartilage, and PRG4 (lubricin) were also downregulated together with MMP1, MMP12 and ADAMTS4 suggesting a role for 5′ isomiR also in extracellular matrix metabolism. Perhaps the role 5′ isomiR plays in cartilage turnover is partly linked to its upregulation of IL6. IL6 is considered to be a strong pro-inflammatory cytokine that enhances inflammatory response, however the role of IL6 in OA is still controversial. Considering some of the effects IL6 has it can also be classified as anti-inflammatory and regenerative. It has been shown that chondrocytes produce high concentrations of IL6 during regeneration, especially osteoarthritic chondrocytes46. Moreover IL6-knockout mice were shown to develop more advanced degenerative changes compared to controls. Also in another study of IL6 deficient mice it was demonstrated that intra-articular injection of IL6 reduced the loss of proteoglycans in the acute phase of chronic joint inflammation and induced the formation of osteophytes.

Heterogenity at the 5′ end as a result of the inconsistent processing by Dicer and Drosha generate 5′ isomiRs with different seed sequences, which affects target recognition and regulation. In the case of canonical miR-140-3p and the 5′ isomiR investigated in this study, one nt change at 5′ end led to the downregulation of 505 more genes compared to canonical miR-140-3p. Interestingly, 94% of the genes downregulated by canonical miR-140-3p were also downregulated by 5′ isomiR. Perhaps the two different seed sequences bind to different target sequences on the 3′ UTR of the same mRNAs. More intriguing is the observation that many genes are downregulated in common between the 5′ and 3′ isomiRs, but not by the canonical sequence. These two isomiRs have different seed sequences, but share the remaining sequence including the additional two 3′ nt, where they differ from the canonical miR-140-3p. This suggests defining roles for parts other than the seed sequence in target determination. Perhaps this includes the involvement of the so-called supplemental region (nucleotides ˜13-16) of the miRNA that supplements seed interactions. It has also been reported that modifications at the 3′ end are associated with miRNA processing and stability. One could speculate whether this particular modification at the 3′ end of the 3′ isomiR enhanced the isomiR's stability leading to a wider target spectrum compared to the canonical sequence despite the identical seed region. The usage of primary cells is both a strength and a limitation of this study. Natural donor variability can sometimes be considerable, as seen in FIGS. 3 and 5. Cell lines, in contrast, tend to exhibit less data variation. Still, despite the donor variability seen using primary cells, we believe they give a more realistic image of the changes induced by the introduction of miRNAs. This may be particularly important as the 5′ isomiR may be a candidate for therapeutic interventions.

Another limitation is the use of stringent statistical tools in the analysis of big data sets. To avoid false discoveries, stringent compensatory measures are used which may lead to false negative results. The downregulation of a number of genes in the IFNA/B cascade and all the HLA class II genes led us to question the apparently unchanged concentrations of IFNA, IFNB and CIITA following miRNA transfection. RT-qPCR analysis showed that IFNB was downregulated by all three sequences, IFNA was not detected, and CIITA was downregulated by all, but most strongly by the 5′ sequence. Also IFNG is an example of a gene disqualified from significance due to donor variation. RNA-seq showed that only the 5′ isomiR downregulated IFNG, while RT-qPCR confirmed its downregulation by 5′ isomiR as well as by the other two miRNAs. It is therefore important to supplement such big data analysis with more sensitive approaches for validation.

### Example 2

Animal Study to Determine the Anti-Inflammatory Effect of miR-140-3p 5′ isomiR

Rupture of the anterior cruciate ligament (ACL) of the right knee was performed in 15 mice. One day prior to the rupture, 3 mice were injected with 5 nmol of an agomiR version of the miR-140-3p 5′ isomiR in a volume of 10 μl. The agomiR has the same sequence as the 5′ isomiR, but is modified by attachment of cholesterol to improve the in vivo transfection efficicacy. Another 3 mice were injected with the 5′ isomiR agomiR plus a tranfection enhancing compound called Invivofectamine. The Invivofectamine 3.0 Reagent is an animal origin-free lipid nanoparticle designed for high efficiency in vivo delivery of siRNA and miRNA, and is produced by ThermoFisher Scientific. The remaining 9 mice constituted different control groups. The animals were sacrificed on day 3, at a time when the inflammatory response is at its peak. The knees were dissected free from the rest of the animals and examined to determine the levels of a number of mRNAs encoding molecules essential for inflammatory responses (Il1b, Zbp1, Irf2, Ifnb1 and Ifng), expression of MHC class II molecules (Ciita) and degradation of articular cartilage (Adamts4).

The results are expressed as percent expression in knees injected with the 5′ isomiR relative to knees injected with an irrelevant control sequence, without or with Invivofectamine. The PCR results are from three animals, each gene analysed in technical triplicates, and calculated relative to the GAPDH endogenous control.

| | 5′ isomir | 5′ isomir agomiR + |
|---|---|---|
| Gene analysed | agomiR only | Invivofectamine |
| Il1b | 56% | 119% |
| Zbp1 | 38% | 77% |
| Irf2 | 68% | 139% |
| Ifnb1 | 45% | Values too low to be reliable |
| Ifng | 39% | 100% |
| Ciita | 75% | 209% |
| Adamts4 | 69% | 63% |
| Average for all genes | 56% | 118% |

The expression of these molecular markers in several tissues of the knee joint in the control mice shows that non-surgical rupture of the ACL causes recruitment of inflammatory cascades. It has previously been shown that this will lead to the development of osteoarthritis in 8-10 weeks following the rupture of ACL.

The downregulation of the inflammation markers by the 5′ isomiR, in several cases to less than half of that observed in control mice, confirms findings made in our in vitro model of osteoarthritis and strongly suggests that the 5′ isomiR will have a clinical effect against osteoarthritis and other inflammation-mediated diseases.

The lack of effect seen in the experiments where Invivofectamine was used may be due to the inflammation-inducing effect of lipid nanoparticles, also previously shown in vitro by our research group.

1-17. (canceled)

18. Oligonucleotide composition comprising one or more nucleotides, or salts thereof, wherein the one or more oligonucleotides is at least 80% identical to a sequence selected from the group consisting of SEQ ID NOs:1, 2 and 3 with the proviso that the oligonucleotides comprise the seed sequence CCACAGG (SEQ ID NO:6) situated at positions 2-8 from the 5′ end of the oligonucleotide.

19. (canceled)

20. Oligonucleotide composition of claim 18, wherein the one or more oligonucleotides consist of from 15 to 40 linked nucleobases.

21. Oligonucleotide composition of claim 18, wherein the one or more oligonucleotides are double-stranded and comprise at least one phosphorothioate linkage and at least one 2′-Fluoro or 2′-Methoxy base.

22. Oligonucleotide composition of claim 18, wherein the one or more oligonucleotides are RNA oligonucleotides.

23. Oligonucleotide composition of claim 18, wherein the oligonucleotide is an agomir.

24. (canceled)

25. A sterile pharmaceutical composition suitable for intra-articular administration comprising an oligonucleotide selected from the group consisting of SEQ ID NOs:1, 2 and 3, wherein the oligonucleotide is double-stranded and comprises at least one phosphorothioate linkage and at least one 2′-Fluoro or 2′-Methoxy base.

26. (canceled)

27. A method of treating an inflammatory disease in subject in need thereof comprising administering to the subject an oligonucleotide that is at least 80% identical to any one of SEQ ID NOs:1, 2 or 3 with the proviso that the oligonucleotides comprise the seed sequence CCACAGG (SEQ ID NO:6) situated at positions 2-8 from the 5′ end of the oligonucleotide.

28. The method of claim 27, wherein the inflammatory disease is selected from the group consisting of inflammatory HLA-related immune diseases, inflammatory arthritic diseases, and inflammatory auto-immune diseases.

29. The method of claim 27, wherein the oligonucleotide is selected from the group consisting of SEQ ID NOs:1, 2 and 3.

30. The method of claim 27, wherein the oligonucleotide consists of from 15 to 40 linked nucleobases.

31. The method of claim 27, wherein the oligonucleotide is an RNA oligonucleotide.

32. The method of claim 27, wherein the oligonucleotide is double-stranded and comprises at least one phosphorothioate linkage and at least one 2′-Fluoro or 2′-Methoxy base.

33. The method of claim 27, wherein the oligonucleotide is an agomir.

34. The method of claim 33, wherein the agomir is double-stranded and comprises an antisense strand having 5′ and 3′ terminals, wherein the antisense strand has a cholesterol-modified 3′ terminal, a plurality of phosphorothioate internucleoside linkages at the 5′ and 3′ terminals, and a plurality of 2′ OMe modified bases.

35. The method of claim 27, wherein the inflammatory disease is osteoarthritis.

36. The method of claim 27, wherein the oligonucleotide is administered intra-articularly.

37. The method of claim 27, wherein the inflammatory disease is osteoarthritis and wherein the oligonucleotide is administered intra-articularly.

38. The method of claim 27, wherein the inflammatory disease is rheumatoid arthritis.

39. The method of claim 27, wherein the oligonucleotide is administered systemically.

40. The method of claim 27, wherein the inflammatory disease is rheumatoid arthritis and wherein the oligonucleotide is administered systemically.
